(19) United States
(12) Patent Application Publication
(10) Pub. No.: US 2014/0303243 A1
(43) Pub. Date: Oct. 9, 2014

(54) Genetic Alterations Associated with Autism and the Autistic Phenotype and Methods of Use Thereof for the Diagnosis and Treatment of Autism

(71) Applicant: The Children's Hospital of Philadelphia, Philadelphia, PA (US)

(72) Inventors: Hakon Hakonarson, Malvern, PA (US); Tara Wenger, Philadelphia, PA (US); Charlly Kao, Philadelphia, PA (US); Dexter Hadley, Philadelphia, PA (US); Zhi-liang Wu, Fort Washington, PA (US); Joseph Glessner, Mullica Hill, NJ (US)

(21) Appl. No.: 14/292,480

(22) Filed: May 30, 2014

Related U.S. Application Data
(63) Continuation-in-part of application No. 14/131,359, filed on Jul. 10, 2014.
(60) Provisional application No. 61/646,971, filed on May 15, 2012.

(51) Int. Cl. C12Q 1/68

(52) U.S. Cl. CPC C12Q 1/6883
USPC 514/455; 435/6.11

(57) ABSTRACT

Compositions and methods for the detection and treatment of autism and autistic spectrum disorder are provided.

This application is a continuation in part application of U.S. patent application Ser. No. 14/131,359, filed Jan. 7, 2014 which is a §371 application of PCT/US12/45959 filed Jul. 9, 2012, which in turn claims priority to U.S. Provisional Application Nos. 61/505,352 and 61/646,971 filed Jul. 7, 2011 and May 15, 2012 respectively, the entire contents of each being incorporated by reference herein as though set forth in full.

Pursuant to 35 U.S.C. '202(c) it is acknowledged that the U.S. Government has certain rights in the invention described, which was made in part with funds from the National Institutes of Health, Grant Numbers NIH T32 GM008628, RC2 MH089924, NIHD070454 and NIMH87636.

### FIELD OF THE INVENTION

This invention relates to the fields of genetics and the diagnosis and treatment of autism and autism spectrum disorders.

### BACKGROUND OF THE INVENTION

Several publications and patent documents are cited throughout the specification in order to describe the state of the art to which this invention pertains. Each of these citations is incorporated herein by reference as though set forth in full.

Autism (MIM [209850]) is a severe and relatively common neuropsychiatric disorder characterized by abnormalities in social behavior and communication skills, with tendencies towards patterns of abnormal repetitive movements and other behavior disturbances. Current prevalence estimates are ˜0.2% of the population for autism and 0.9% of the population for ASDs (MMWR Surveill Summ. 2009). Globally, males are affected four times as often as females². As such, autism poses a major public health concern of unknown cause that extends into adulthood and places an immense economic burden on society. The most prominent features of autism are social and communication deficits. The former are manifested in reduced sociability (reduced tendency to seek or pay attention to social interactions), a lack of awareness of social rules, difficulties in social imitation and symbolic play, impairments in giving and seeking comfort and forming social relationships with other individuals, failure to use nonverbal communication such as eye contact, deficits in perception of others' mental and emotional states, lack of reciprocity, and failure to share experience with others. Communication deficits are manifested as a delay in or lack of language, impaired ability to initiate or sustain a conversation with others, and stereotyped or repetitive use of language. Autistic children have been shown to engage in free play much less frequently and at a much lower developmental level than peers of similar intellectual abilities. Markers of social deficits in affected children appear as early as 12-18 months of age, suggesting that autism is a neurodevelopmental disorder. It has been suggested that autism originates in developmental failure of neural systems governing social and emotional functioning. Although social and cognitive development are highly correlated in the general population, the degree of social impairment does not correlate well with IQ in individuals with autism. The opposite is seen in Down's syndrome and Williams syndrome, where social development is superior to cognitive function. Both examples point to a complex source of sociability.

The etiology of the most common forms of autism is still unknown. In the first description of the disease in 1943, Kanner suggested an influence of child-rearing practices on the development of autism, after observing similar traits in parents of the affected children. While experimental data fail to support several environmental hypotheses, there has been growing evidence for a strong genetic influence on this disorder. The rate of autism in siblings of affected individuals was shown to be 8.6%, a 215 fold increase over the general population (Ritvo et al. (1989) Am J Psychiatry 146(8):1032-6.). Twin studies have demonstrated significant differences in monozygotic and dizygotic twin concordance rates, the former concordant in 60% of twin pairs, with most of the non-autistic monozygotic co-twins displaying milder related social and communicative abnormalities. Social, language and cognitive difficulties have also been found among relatives of autistic individuals in comparison to the relatives of controls. The heritability of autism has been estimated to be >90%.

The genetic basis of autism has been extensively studied in the past decade using three complementary approaches: cytogenetic studies; linkage analysis, and candidate gene analysis see for a review (Freitag, C. M. et al., (2010) Eur Child Adolesc Psychiatry 19(3):169-78; Vorstman et al., (2006) Mol. Psychiatry 11:18-28; Veenstra-VanderWeele and Cook, (2004) Mol. Psychiatry 9: 819-32). Searches for chromosomal abnormalities in autism have revealed terminal and interstitial deletions, balanced and unbalanced translocations, and inversions on a large number of chromosomes, with abnormalities on chromosomes 15,7, and X being most frequently reported. The importance of the regions indicated by cytogenetic studies was evaluated by several whole genome screens in the multiplex autistic families (International Molecular Genetic Study of Autism Consortium, 1998). Strong and concordant evidence for the presence of an autism susceptibility locus was obtained for chromosome 7q; moderate evidence was obtained for loci on chromosomes 15q, 16p, 19p, and 2q; and the majority of the studies find no support for linkage to the X chromosome (Lamb et al, (2005) Med Genet. 42: 132-137; Lord et al, (2000) Autism Dev Disord. 30:205-223; Muhle et al., (2004) Pediatrics 113(5): e472-86). The AGRE sample provided the strongest evidence for loci on 17q and 5p (Yonan et al., (2003) Am J Hum Genet. 73:886-97). Numerous candidate gene studies in autism have focused on a few major candidates with respect to their location or function (reviewed in Veenstra-VanderWeele et al 2004, supra). Jamain et al., ((2003) Nat Genet. 34:27-9), reported rare nonsynonymous mutations in the X-linked genes encoding neuroligins, specifically NLGN3 and NLGN4, in linkage regions associated with ASD. Other evidence for a genetic basis of autistic endophenotypes comes from the study of disorders that share phenotypic features that overlap with autism such as Fragile X and Rett syndrome.

Many emerging theories of autism focus on changes in neuronal connectivity as the potential underlying cause of these disorders. Imaging studies reveal changes in local and global connectivity (Just et al., (2004) Brain 127: 1811-1821; Herbert et al., (2005) Ann Neurol 55(4): 530-40) and developmental studies of activity-dependent cortical development suggest that autism might result from an imbalance of inhibitory and excitatory synaptic connections during development (Rubenstein and Merzenich, (2004; Genes Brain Behav 2(5): 255-67). The fundamental unit of neuronal connectivity is the synapse; thus, if autism is a disorder of neuronal connectivity, then it can likely be understood in neuronal terms as a disorder of synaptic connections. Indeed, genetic studies reveal that mutations in key proteins involved in synaptic development and plasticity, such as neuroligins, FMRP and MeCP2 are found in individuals with autism and in two forms of mental retardation with autistic features, specifically Fragile-X and Rett's syndrome (Jamain et al, 2003, supra, O'Donnell and Warren, (2002) Annu Rev Neurosci 25: 315-38). Thus the pursuit of linkage between genetic anomalies and (endo)phenotypes at the neuronal level appears both warranted and fruitful. Furthermore, such neuronal connectivity anomalies, revealed, for example, by direct white matter tractography, or by observable delays in characteristic electrical activity, can be directly linked to behavioral and clinical manifestations of ASD, allowing these neuron-level phenotypes to be interpreted as neural correlates of behavior.

Overall, the linkage analysis studies conducted to date and discussed above have achieved only limited success in identifying genetic determinants of autism due to numerous reasons, among others the generic problem that the linkage analysis approach is generally poor in identifying common genetic variants that have modest effects (Hirschhorn and Daly, (2005) Nat Rev Genet 6(2): 95-108). This problem is highlighted in autism, a spectrum disorder wherein the varied phenotypes are determined by the net result of interactions between multiple genetic and environmental factors and, in which, any particular genetic variant that is identified is likely to contribute little to the overall risk for disease.

In one of the first studies to report an association of de novo copy number variations (CNVs) with autism (Science (2007) Apr. 20 ;316(5823):445-9), Sebat and colleagues suggest that CNVs may underlie certain cases of the disease. Indeed, the importance of their findings have been recapitulated in other work (Pinto et al, Nature. 2010 Jul. 15 ;466(7304):368-72.; Glessner et al Nature. 2009 May 28 ;459(7246):569-73) suggesting that CNVs may at least account for a small percentage of the genetic variation of the ASDs. However, these genetic defects are rare and collectively only explain a small proportion of the genetic risk for autism, thus suggesting the existence of additional genetic loci but with unknown frequency and effect size.

### SUMMARY OF THE INVENTION

The present inventors have performed genome wide association study on several large patient cohorts and have successfully identified a number of target genes harboring copy number variations associated with autism and ASD. Thus, in accordance with the present invention, kits are provided for performance of a method for detecting a propensity for developing autism or autistic spectrum disorder is provided. An exemplary kit comprises means for obtaining a sample from a patient and testing the sample for the presence or absence of at least one deletion containing CNV in a target polynucleotide, wherein if the CNV is present, the patient has an increased risk for developing autism and/or autistic spectrum disorder. In a preferred embodiment, the deletion containing CNV is selected from the group of CNVs provided in Table II. In another embodiment, the kit includes reagents for performing the step of detecting the presence of said CNV and further comprises specific reagents for performing a process selected from the group consisting of detection of specific hybridization, measurement of allele size, restriction fragment length polymorphism analysis, allele-specific hybridization analysis, single base primer extension reaction, and sequencing of an amplified polynucleotide.

In another aspect, the present invention provides a method for identifying agents which alter neuronal signaling and/or morphology. An exemplary method entails providing cells expressing at least one CNV listed in Table 2 and cells which express the cognate wild type sequences corresponding to the CNV containing sequence, contacting both cell types with a test agent and analyzing whether the agent alters neuronal signaling and/or morphology of cells comprising the CNV relative to those which lack the genetic alteration, thereby identifying agents which alter neuronal signaling and morphology in CNV containing cells. In cases where the CNV is a deletion, vectors encoding such CNVs contain nucleic acids flanking the affected region of deletion of a suitable length, such that cloning and transformation of cells with the CNV containing nucleic acid is possible.

Also provided is a method of treating autism or ASD in a human subject determined to have at least copy number variation (CNV) associated with an autistic or ASD phenotype, said at least one CNV being selected from the group consisting of CNVs set out in Table 2, the method comprising administering to said human subject a therapeutically effective amount of at least one agent which is known to be efficacious in the signaling pathway adversely affected by the presence of said CNV. In a preferred embodiment, patients are tested for the presence or absence of at least one CNV containing gene is selected from the group consisting of ATP10A, GABRA5, GABRB3, GABRG3, GGTLC2, HBII-52-45, HBII-52-46, IPW, LOC648691, LOC96610, MAGEL2, MIR650, MKRN3, NCRNA00221, NDN, OCA2, OR4S2, PAR-SN, PAR1, PARS, POM121L1P, PRAME, SNORD107, SNORD108, SNORD109A, SNORD109B, SNORD115-11, SNORD115-29, SNORD115-36, SNORD115-43, SNORD115-44, SNORD115-48, SNORD64, SNRPN, SNURF, UBE3A, ZNF280A, ZNF280B. In yet another embodiment, the CNV is determined to reside in a gene important for GABA signaling and the agent is listed in Table 3 or Table 4. In a particularly preferred embodiment, the CNV alters GABA signaling and the agent is topiramide.

### BRIEF DESCRIPTION OF THE DRAWINGS

FIG. 1: The design of the present study is shown. In this two-stage design, 2076 cases vs 4754 controls were used in the discovery cohort (Stage 1), and 1159 cases vs 2546 controls were used for a replication cohort (Stage 2). All samples used passed minimal quality control metrics, but the default quality calls of PennCNV were used to discriminate the discovery cohort (best quality) from the replication cohort (lesser quality.)

FIG. 2: A graph showing the classes of gene pathways, which are disrupted by the ASD-related CNVRs disclosed herein. All genes with exons disrupted by replicated CNVRs were submitted to Ingenuity to ascertain significance of pathway enrichment.

FIG. 3: A schematic of a first-degree interactome of the GABAR-A family highlighting copy number defects enriched in cases (red) vs controls (blue).

FIG. 4: Test and treat model for targeting therapeutics to specific pathways defective in disease. The generic test and treat model is shown in black where a molecular diagnostic is used to genetically define a population with defective pathways that are likely to benefit from a targeted intervention. Examples of trastuzumab as a targeted intervention for HER2 specific breast cancer is shown in blue as well as an extrapolation of behavioral programs and novel therapeutics that are being developed to target ASDs due to defective GABAR-A pathways in red.

FIG. 5: Significance of CNVRs by GWAS of ASDs in European-derived or African-derived populations. The Manhattan plots shows the −log 10 transformed P value of association for each CNVR along the genome. Adjacent chromosomes are shown in alternating red and blue colors. The regions discovered in Europeans (P 0.0001) that replicated in Africans (P<=0.001) are highlighted with black arrows—labeled by chromosome band. GWAS of 4,634 cases vs 4,726 controls in Europeans is shown on top and GWAS of 312 cases vs 4,173 controls in Africans is shown below.

FIG. 6: Enrichment of optimal CNVRs across mGluR network of genes. Nodes of the network are labeled with their gene names, with red and green representing deletions and duplications respectively, while grey nodes lack CNV data. Dark and light colors represent enrichment in cases and controls respectively. The genes defining the network are showed as diamonds, while all other genes are shown as circles. Blue lines indicate evidence of interaction.

FIG. 7: Enrichment of optimal CNVRs across CALM1 network. The first degree directed interaction network defined by CALM1 is shown.

FIG. 8: Diagram of study design. Children with ASD and gene changes in mGluR network (mGluR+ASD) were more likely to have Syndromic ASD compared to children with ASD without abnormalities of mGluR network genes (mGluR-ASD). P<0.0001.

### DETAILED DESCRIPTION OF THE INVENTION

Epidemiologic studies have convincingly implicated genetic factors in the pathogenesis of autism, a common neuropsychiatric disorder in children, which presents with variable phenotype expression that extends into adulthood. Several genetic determinants have already been reported to be associated with ASD, including many rare de novo copy number variants (CNVs) that harbor small genomic deletions and insertions. These genetic alterations may account for a small subset of the phenotypic manifestation of the disease. Implicated genomic regions appear to be highly heterogeneous with variations reported in several genes, including NRXN1, NLGN3, SHANK3 and AUTS2 to date.

Predicting an individual's genomic risk for disease can facilitate the development of new interventions and streamline therapeutic approaches. To identify likely functional CNVs, we combined various large cohorts of autistic patients with a large number of neurologically normal controls to analyze over 3K affected cases and 7K controls. In a two-stage genome-wide association design, we uncovered 266 genome-wide statistically significant (combined P<=2.76×10⁻⁸) distinct CNV regions (CNVR).

The 38 genes with exons disrupted by these robust CNVRs are most enriched in gene networks impacting neurological disease, behavior and developmental disorders. GABAR-A receptor signaling was the most significant disrupted canonical pathway in ASD where case-enriched defects in GABRA5, GABRB3, and GABRG3 genes were identified. Moreover, network analysis of the first-degree gene interactome of the GABAR-A receptor family suggests that ASD cases are significantly enriched for such pathway defects (P<=2.1×10⁻²¹, OR=9.9) when compared with neurologically normal controls.

Taken together, the CNVRs we have identified impact multiple novel genes and signaling pathways, including genes involved in GABAR-A signaling, that can provide important targets for therapeutic intervention.

Since drugs must compete with endogenous small molecules for protein binding, many successful drugs target large gene families with multiple drug binding sites. In Example III, we search for defective gene family interaction networks (GFINs) in 6,742 patients with the ASDs relative to 12,544 neurologically normal controls, to find potentially additional genetic targets that may be amenable for drug therapy. We find significant enrichment of structural defects (P<=2.40×10⁻⁹, 1.8-fold enrichment) in the metabotropic glutamate receptor (GRM) GFIN, described in Example I and previously observed to impact attention deficit hyperactivity disorder (ADHD) and schizophrenia. Also, the MXD-MYC-MAX network of genes, previously implicated in cancer, is significantly enriched (P<=3.83×10⁻²³, 2.5-fold enrichment), as is the calmodulin 1 (CALM1) gene interaction network (P<=4.16×10⁻⁴, 14.4-fold enrichment) which regulates voltage independent calcium-activated action potentials at the neuronal synapse. In conclusion, we find multiple defective gene family interactions underlie autism, which provide many novel translational targets for therapeutic interventions.

### DEFINITIONS

A “copy number variation (CNV)” refers to the number of copies of a particular gene in the genotype of an individual. CNVs represent a major genetic component of human phenotypic diversity. Susceptibility to genetic disorders is known to be associated not only with single nucleotide polymorphisms (SNP), but also with structural and other genetic variations, including CNVs. A CNV represents a copy number change involving a DNA fragment that is ˜1 kilobases (kb) or larger (Feuk et al. 2006a). CNVs described herein do not include those variants that arise from the insertion/deletion of transposable elements (e.g., ˜6-kb KpnI repeats) to minimize the complexity of future CNV analyses. The term CNV therefore encompasses previously introduced terms such as large-scale copy number variants (LCVs; lafrate et al. 2004), copy number polymorphisms (CNPs; Sebat et al. 2004), and intermediate-sized variants (ISVs; Tuzun et al. 2005), but not retroposon insertions.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with autism. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated.

The term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any autism specific marker gene or nucleic acid, but does not hybridize to other nucleotides. Also polynucleotide which “specifically hybridizes” may hybridize only to a neurospecific specific marker, such an autism-specific marker shown in the Table contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log[Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence and may or may not comprise a detectable label. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application, also it may or may not be detectably labeled. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product. Probes and primers having the appropriate sequence homology which specifically hybridized to CNV containing nucleic acids are useful in the detecting the presence of such nucleic acids in biological samples.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the autism specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the autism specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably an autism specific marker molecule, such as a marker shown in the tables provided below. Samples may include but are not limited to cells, body fluids, including blood, serum, plasma, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP and/or CNV containing nucleic acids described herein or their encoded proteins. Agents are evaluated for potential biological activity by inclusion in screening assays described hereinbelow.

### Methods of Using Autism-Associated CNVs and/or SNPs for Diagnosing a Propensity for the Development of Autism and Autistic Spectrum Disorders

Autism-related-CNV and/or SNP containing nucleic acids, including but not limited to those listed in the Table provided below may be used for a variety of purposes in accordance with the present invention. Autism-associated CNV/SNP containing DNA, RNA, or fragments thereof may be used as probes to detect the presence of and/or expression of autism specific markers. Methods in which autism specific marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

Further, assays for detecting autism-associated CNVs/SNPs may be conducted on any type of biological sample, including but not limited to body fluids (including blood, urine, serum, gastric lavage), any type of cell (such as brain cells, white blood cells, mononuclear cells) or body tissue. Such detection methods can include for example, southern and northern blotting, RFLP, direct sequencing and PCR amplification followed by hybridization of amplified products to a microarray comprising reference nucleic acid sequences.

From the foregoing discussion, it can be seen that autism-associated CNV/SNP containing nucleic acids, vectors expressing the same, autism CNV/SNP containing marker proteins and anti-Autism specific marker antibodies of the invention can be used to detect autism associated CNVs/SNPs in body tissue, cells, or fluid, and alter autism SNP containing marker protein expression for purposes of assessing the genetic and protein interactions involved in the development of autism. In most embodiments for screening for autism-associated CNVs/SNPs, the autism-associated CNV/SNP containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art. Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus any of the aforementioned techniques may be used to detect or quantify autism-associated CNV/SNP marker expression and accordingly, diagnose autism or an autism spectrum disorder.

### Kits and Articles of Manufacture

Any of the aforementioned products can be incorporated into a kit which may contain a autism-associated CNV/SNP specific marker polynucleotide or one or more such markers immobilized on a Gene Chip, an oligonucleotide, a polypeptide, a peptide, an antibody, a label, said label being detectable and optionally, operably linked to an oligonucleotide, polypeptide or antibody marker, or reporter, a pharmaceutically acceptable carrier, a physiologically acceptable carrier, instructions for use, a container, a vessel for administration, an assay substrate, or any combination thereof.

In a preferred embodiment, the kit contains reagents for identifying nucleic acids present in a biological sample with harbor nucleic acids comprising the genetic alterations described herein. In the case where the CNV is a deletion, probes or primers are designed to flank the affected region in order to assess whether the CNV is present or absent.

### Methods of Using Autism-Associated Cnvs/Snps for Development of Therapeutic Agents

Since the CNVs and SNPs identified herein have been associated with the etiology of autism, methods for identifying agents that modulate the activity of the genes and their encoded products containing such CNVs/SNPs should result in the generation of efficacious therapeutic agents for the treatment of a variety of disorders associated with this condition.

As can be seen from the data provided in Table 1, several chromosomes contain regions which provide suitable targets for the rational design of therapeutic agents which modulate their activity. Small peptide molecules corresponding to these regions may be used to advantage in the design of therapeutic agents which effectively modulate the activity of the encoded proteins.

Molecular modeling should facilitate the identification of specific organic molecules with capacity to bind to the active site of the proteins encoded by the CNV/SNP containing nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered autism associated gene. These host cell lines or cells are defective at the polypeptide level. The host cell lines or cells are grown in the presence of drug compound. The rate of cellular metabolism, alterations in cellular morphology and/or receptor signaling of the host cells is measured to determine if the compound is capable of altering any of these parameters in the defective cells. Host cells contemplated for use in the present invention include but are not limited to bacterial cells, fungal cells, insect cells, mammalian cells, and plant cells. The autism-associated CNV/SNP encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1N5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF), the Thy-1 promoter, the hamster and mouse Prion promoter (MoPrP), and the Glial fibrillar acidic protein (GFAP) for the expression of transgenes in glial cells.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the autism-associated CNVs/SNPs of the present invention or functional fragments thereof provide a system in which to screen potential compounds or agents for the ability to modulate the development of autism. Thus, in one embodiment, the nucleic acid molecules of the invention may be used to create recombinant cell lines for use in assays to identify agents which modulate aspects of cellular metabolism associated with neuronal signaling and neuronal cell communication and structure. Also provided herein are methods to screen for compounds capable of modulating the function of proteins encoded by CNV/SNP containing nucleic acids.

Another approach entails the use of phage display libraries engineered to express fragment of the polypeptides encoded by the CNV/SNP containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the therapeutic.

Thus, one may design drugs which have, e.g., improved polypeptide activity or stability or which act as inhibitors, agonists, antagonists, etc. of polypeptide activity. By virtue of the availability of CNV/SNP containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of autism-associated CNV/SNP containing nucleic acids enables the production of strains of laboratory mice carrying the autism-associated CNVs/SNPs of the invention. Transgenic mice expressing the autism-associated CNV/SNP of the invention provide a model system in which to examine the role of the protein encoded by the SNP containing nucleic acid in the development and progression towards autism. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic and neuronal processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of autism-associated CNV/SNP containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated autism-associated CNV/SNP genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing autism-associated SNP containing nucleic acid as a targeted insertional cassette provides means to detect a successful insertion as visualized, for example, by acquisition of immunoreactivity to an antibody immunologically specific for the polypeptide encoded by autism-associated SNP nucleic acid and, therefore, facilitates screening/selection of ES cells with the desired genotype.

As used herein, a knock-in animal is one in which the endogenous murine gene, for example, has been replaced with human autism-associated CNV/SNP containing gene of the invention. Such knock-in animals provide an ideal model system for studying the development of autism.

As used herein, the expression of a autism-associated CNV/SNP containing nucleic acid, fragment thereof, or an autism-associated CNV/SNP fusion protein can be targeted in a “tissue specific manner” or “cell type specific manner” using a vector in which nucleic acid sequences encoding all or a portion of autism-associated CNV/SNP are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

The nucleic acid sequence encoding the autism-associated CNV/SNP of the invention may be operably linked to a variety of different promoter sequences for expression in transgenic animals. Such promoters include, but are not limited to a prion gene promoter such as hamster and mouse Prion promoter (MoPrP), described in U.S. Pat. No. 5,877,399 and in Borchelt et al., Genet. Anal. 13(6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter; a neuronal-specific platelet-derived growth factor B gene promoter; and Glial fibrillar acidic protein (GFAP) promoter for the expression of transgenes in glial cells.

Methods of use for the transgenic mice of the invention are also provided herein. Transgenic mice into which a nucleic acid containing the autism-associated CNV/SNP or its encoded protein have been introduced are useful, for example, to develop screening methods to screen therapeutic agents to identify those capable of modulating the development of autism.

### Pharmaceuticals and Peptide Therapies

The elucidation of the role played by the autism associated CNVs/SNPs described herein in neuronal signaling and brain structure facilitates the development of pharmaceutical compositions useful for treatment and diagnosis of autism. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following materials and methods are provided to facilitate the practice of the present invention.

Study design & Quality Control

PennCNV was used to define CNVs across all genotyped samples. To control for potential chip-to-chip bias from the mixed SNP content introduced by genotyping across multiple chips types, only CNV calls from the 550K joint SNPs across the 550K, 610K, 660K, and 1M Illumina chips were considered. Low quality samples were excluded on a per sample basis if:

1. # CNVs>100

2. SD LRR>0.3

3.|GCWF|>0.02

### Statistical Analysis

For each stage of analysis, the genome was segmented into CNV regions (CNVRs) that define unambiguous sets of cases and controls impacted by CNVs which facilitates the immediate identification of “core” CNV genomic regions. These CNVRs were tested for association by Fisher's exact test in a two-stage design with an alpha of P<=0.01 after correcting for multiple tests.

### Network Analysis

Ingenuity pathway analysis was used to look for enrichment in networks and canonical pathways among genes with exons disrupted by replicated CNVRs. Fisher's exact test was used to gauge enrichment of the first order interactome of GABAR family of genes, as well as a test of 1000 random permutations of case/control labels.

The following examples are provided to illustrate certain embodiments of the invention. They are not intended to limit the invention in any way.

### Example I

The ability to quantify individual's genomic risk for disease can facilitate the development of new interventions and improve medical practice. Many rare Copy

Number Variants (CNVs) that harbor small genomic deletions and insertions have been described in the autism spectrum disorders (ASD). To identify these likely functional elements, we combined various large cohorts of autistic patients with a large number of neurologically normal controls to analyze over 3K affected cases and 7K controls. In a two-stage genome-wide association design, we uncovered 266 genome-wide statistically significant (combined P<=2.76×10⁻⁸) distinct CNV regions (CNVR).

The 38 genes with exons disrupted by these robust CNVRs are most enriched in gene networks impacting neurological disease, behavior and developmental disorder. GABAR-A receptor signaling was found to be the most significant canonical pathways disrupted in ASD because case-enriched defects in GABRA5, GABRB3, and GABRG3 genes. Moreover, network analysis of the first-degree gene interactome of the GABAR-A receptor family suggests that ASD cases are significantly enriched for pathway defects (P<=2.1×10⁻²¹, OR=9.9) when compared with neurologically normal controls.

Taken together, the CNVRs we have identified impact multiple novel genes and signaling pathways, including genes involved in GABAR-A signaling, that may be important for new therapeutic development.

### Results

In all, 3871 unrelated cases were compared to 7768 controls. Samples were sourced from five independent sites, and were distributed as follows:

TABLE 1

| Cohort | # SNPs measured | #cases | #controls |
|---|---|---|---|
| Site #1 | 550K | 926 | |
| Site #2 | 550K | 1237 | |
| Site #3 | 1M | 799 | |
| Site #4 | 610K + 660K | 266 | |
| Site #5 | 550K + 610K + 660K | 643 | 7768 |
| Total | | 3871 | 7768 |

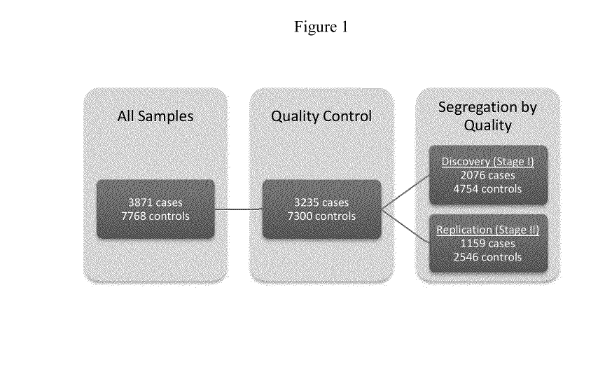

In all, 3225 cases and 7300 controls passed quality control and were used for CNV analysis. These individuals were segregated into a discovery (stage 1) and replication (stage 2) cohort based on the default quality calls of PennCNV. In this two-stage design, 2076 cases vs 4754 controls were used in the discovery cohort, and 1159 cases vs 2546 controls were used for a replication cohort. See FIG. 1.

In the discovery stage, 353 significant CNVRs (nominal P<=1.8×10⁻⁸) were identified after Bonferroni correction for 550K SNPs used for analysis, and 266 significant CNVRs replicated (nominal P<=2.9×10⁻⁵) after correcting for 353 significant discovery regions tested. The most significantly associated CNVRs highlight some attractive and novel candidate genes for ASD.

Most interesting are the 25 duplications unique to cases in GABRB3-GABA-A receptor, beta 3 (P<=1.42×10⁻¹³, OR=inf). This is an attractive candidate gene as GABA is the main inhibitory neurotransmitter, and it lies within the Prader-Willi/Angelman syndrome critical region (15q11-13), mutations of which have been described in several individuals with autism. Moreover, this was found to be significant across Europeans and African populations (P<=6.44×10⁻⁵ and 1.82×10⁻⁵ respectively); Association between a GABRB3 polymorphism and autism (Buxbaum et al., 2002) as well as GABRA4 & GABRB 1 (Collins et al., 2006). Gabrb3 gene deficient mice exhibit impaired social and exploratory behaviors, deficits in non-selective attention and hypoplasia of cerebellar vermal lobules: a potential model of autism spectrum disorder (DeLorey, Sahbaie, Hashemi, Homanics, & Clark, 2008)

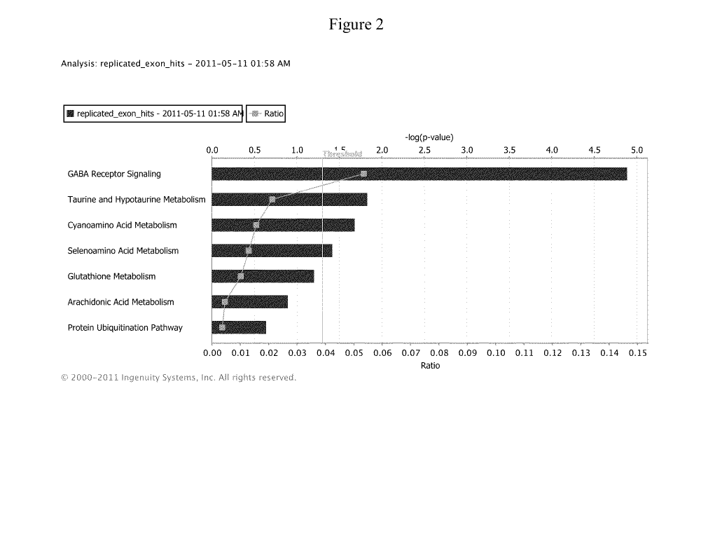

We found 38 genes with exons disrupted by robust CNVRs: ATP10A, GABRA5, GABRB3, GABRG3, GGTLC2, HBII-52-45, HBII-52-46, IPW, LOC648691, LOC96610, MAGEL2, MIR650, MKRN3, NCRNA00221, NDN, OCA2, OR4S2, PAR-SN, PAR1, PARS, POM121L1P, PRAME, SNORD107, SNORD108, SNORD109A, SNORD109B, SNORD115-11, SNORD115-29, SNORD115-36, SNORD115-43, SNORD115-44, SNORD115-48, SNORD64, SNRPN, SNURF, UBE3A, ZNF280A, ZNF280B. These genes are most enriched in gene networks impacting neurological disease, behavior and developmental disorder, and GABAR-A receptor signaling was found to be the most significant canonical pathways disrupted in ASD associated with case-enriched defects in GABRA5, GABRB3, and GABRG3 genes. See FIG. 2 and Table 2.

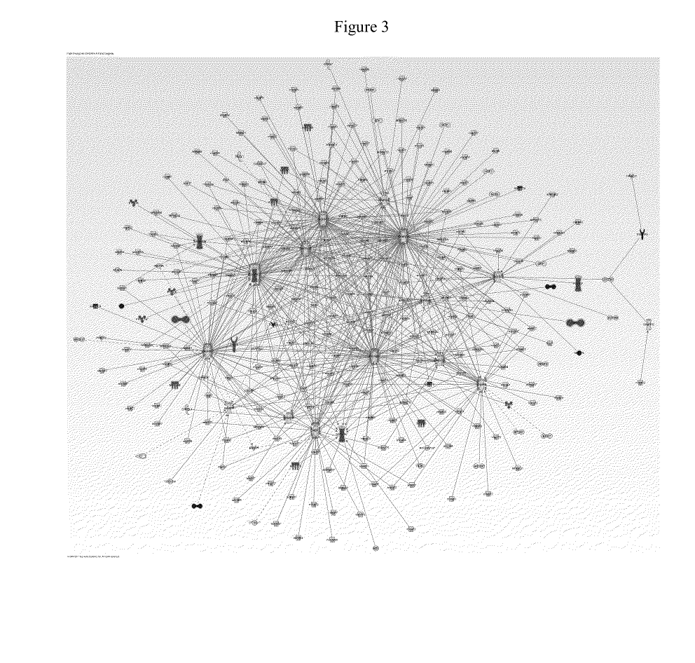

Finally, we defined the first-degree interactome of the GABAR-A family, and a found that ASD cases are significantly enriched for pathway defects in cases when compared with neurologically normal controls. About 3% of cases harbor genetic pathway defects vs 0.03% of controls (P<=2.1×10⁻²¹, OR=9.9), and 17 out of 121 genes enriched in cases (14%) vs 9 out of 121 genes in controls (7%). The network showing genes enriched in cases (red) vs controls (blue) is as shown in FIG. 3.

TABLE 2

| | | | | | | | replica- | | | | |
|---|---|---|---|---|---|---|---|---|---|---|---|
| band | size | numSnp | bestP | bestOr | bestP | discoveryBestP | tionBestP | delCases | delControls | delPval | delOR |
| 7q34 | 1 | 1 | 4.88E−32 | 2.02 | 4.97779E−32 | 1.76E−29 | 3.92E−06 | 614 | 758 | 4.08E−32 | 2.02 |
| 11q11 | 4405 | 2 | 1.46E−18 | 1.58 | 1.45699E−18 | 2.14E−12 | 1.51E−07 | 776 | 1215 | 1.46E−18 | 1.58 |
| 14q32.33 | [symbol] | 1 | 2.61E−34 | 36.05 | 2.61032E−34 | 1.30E−25 | 5.53E−09 | 78 | 5 | 2.61E−34 | 36.05 |
| 15q11.2 | 102655 | 19 | 8.28E−13 | 59.14 | 8.28009E−13 | 1.50E−08 | 8.74E−06 | 3 | 2 | 1.73E−01 | 3.39 |
| 15q11.2 | 72723 | 11 | 2.61E−12 | 56.85 | 2.61199E−12 | 1.50E−08 | 8.74E−06 | 3 | 2 | 1.73E−01 | 3.39 |
| 15q11.2 | 65697 | 7 | 2.61E−12 | 56.85 | 2.61199E−12 | 1.50E−08 | 2.81E−05 | 3 | 2 | 1.73E−01 | 3.39 |
| 15q11.2 | 4052 | 2 | 2.15E−12 | 56.85 | 2.14941E−12 | 1.50E−08 | 2.33E−05 | 4 | 2 | 7.62E−02 | 4.52 |
| 15q11.2 | 141181 | 34 | 2.61E−12 | 56.85 | 2.61199E−12 | 1.50E−08 | 2.81E−05 | 3 | 2 | 1.73E−01 | 3.39 |
| 15q11.2 | 44496 | 211 | 2.15E−12 | 56.85 | 2.14941E−12 | 1.50E−08 | 2.81E−05 | 4 | 2 | 7.62E−02 | 4.52 |
| 15q11.2 | 29599 | 2 | 2.61E−12 | 56.85 | 2.61199E−12 | 1.50E−08 | 2.81E−05 | 1 | 2 | 6.67E−01 | 1.13 |
| 15q11.2-12 | 266225 | 34 | 2.61E−12 | 56.85 | 2.61199E−12 | 1.50E−08 | 2.81E−05 | 3 | 2 | 1.73E−01 | 3.39 |
| 15q12 | 52133 | 28 | 2.61E−12 | 56.85 | 2.61199E−12 | 1.50E−08 | 2.81E−05 | 3 | 2 | 1.73E−01 | 3.39 |
| 15q12 | 5489 | 3 | 1.42E−13 | 21.24 | 1.42231E−13 | 2.01E−09 | 2.81E−05 | 3 | 3 | 2.69E−01 | 2.26 |
| 15q12 | 173668 | 15 | 2.61E−12 | 56.85 | 2.61199E−12 | 1.50E−08 | 2.81E−05 | 3 | 2 | 1.73E−01 | 3.39 |
| 15q12 | 204333 | 8 | 1.42E−13 | 31.86 | 1.42231E−13 | 2.01E−09 | 2.81E−05 | 3 | 2 | 1.73E−01 | 3.39 |
| 15q13.1 | 9266 | 3 | 2.15E−12 | 56.85 | 2.14941E−12 | 1.50E−08 | 2.81E−05 | 4 | 2 | 7.62E−02 | 4.52 |
| 22q11.22 | 9572 | 2 | 2.88E−13 | 33.01 | 2.85729E−13 | 1.69E−08 | 2.81E−05 | 20 | 0 | 5.33E−11 | None |
| 22q11.22 | 359 | 2 | 4.50E−32 | 147.32 | 4.50117E−32 | 1.58E−21 | 2.70E−11 | 64 | 1 | 4.50E−32 | 147.32 |
| 22q11.22 | [symbol] | 1 | 8.12E−34 | 35.57 | 8.12264E−34 | 2.10E−20 | 1.20E−13 | 49 | 0 | 5.82E−26 | None |
| 22q11.22 | 1 | 1 | 6.77E−32 | 40.93 | 6.76528E−32 | 1.94E−19 | 1.16E−11 | 53 | 0 | 4.95E−28 | None |
| 22q11.22 | 3719 | 2 | 4.56E−29 | 36.81 | 4.56859E−29 | 5.84E−20 | 1.05E−10 | 55 | 0 | 4.56E−29 | None |
| 22q11.22 | 5387 | 3 | 3.16E−32 | 77.17 | 3.16442E−32 | 1.75E−20 | 1.20E−13 | 59 | 0 | 3.86E−31 | None |
| 22q11.22 | 32636 | 2 | 5.37E−27 | 64.28 | 5.36793E−27 | 1.94E−19 | 1.15E−11 | 51 | 0 | 5.37E−27 | None |
| 22q11.22 | 18133 | 2 | 1.04E−30 | 73.65 | 1.04248E−30 | 2.41E−58 | 2.32E−10 | 64 | 2 | 1.04E−30 | 73.65 |

| band | size | dupCases | dupControls | dupPval | dupOR | cnvCases | cnvControls | cnvPval | cnvOR | geneHit |
|---|---|---|---|---|---|---|---|---|---|---|
| 7q34 | 1 | 36 | 78 | 4.55E−01 | 1.04E+00 | 650 | 836 | 1.47E−30 | 1.94 | MGAM |
| 11q11 | 4405 | 48 | 313 | 1.00E+00 | 3.36E−01 | 824 | 1528 | 1.75E−07 | 1.29 | OR4S2 |
| 14q32.33 | [symbol] | 13 | 49 | 9.68E−01 | 5.97E−01 | 91 | 54 | 1.16E−15 | 3.88 | MCRNA00221 |
| 15q11.2 | 102655 | 26 | 1 | 8.28E−13 | 5.91E+01 | 29 | 3 | 2.15E−12 | 22.00 | MKRN3 |
| 15q11.2 | 72723 | 25 | 1 | 2.61E−12 | 5.68E+01 | 28 | 3 | 6.39E−12 | 21.24 | MAGEL2 |
| 15q11.2 | 65697 | 25 | 1 | 2.61E−12 | 5.68E+01 | 28 | 3 | 6.39E−12 | 21.24 | NDN |
| 15q11.2 | 4052 | 25 | 1 | 2.61E−12 | 5.68E+01 | 29 | 3 | 2.15E−12 | 22.00 | SNRPN |
| 15q11.2 | 141181 | 25 | 1 | 2.61E−12 | 5.68E+01 | 28 | 3 | 6.39E−12 | 21.24 | PAR-SN SNURF |
| | | | | | | | | | | PAR5 SNRPN |
| | | | | | | | | | | SNORD 107-109 |
| 15q11.2 | 44496 | 25 | 1 | 2.61E−12 | 5.68E+01 | 29 | 3 | 2.15E−12 | 22.00 | IPW PAR1 |
| 15q11.2 | 29599 | 25 | 1 | 2.61E−12 | 5.68E+01 | 26 | 3 | 5.59E−11 | 19.71 | SNORD109 115 |
| | | | | | | | | | | HBII-52 |
| 15q11.2-12 | 266225 | 25 | 1 | 2.61E−12 | 5.68E+01 | 28 | 3 | 6.39E−12 | 21.24 | UBE3A |
| 15q12 | 52133 | 25 | 1 | 2.61E−12 | 5.68E+01 | 28 | 3 | 6.39E−12 | 21.24 | ATP10A |
| 15q12 | 5489 | 25 | 0 | 1.42E−13 | None | 28 | 3 | 6.39E−12 | 21.24 | GABRB3 |
| 15q12 | 173668 | 25 | 1 | 2.61E−12 | 5.68E+01 | 28 | 3 | 6.39E−12 | 21.24 | GABRA5 |
| | | | | | | | | | | GABRG3 |
| 15q12 | 294333 | 25 | 0 | 1.42E−13 | None | 28 | 2 | 8.76E−13 | 31.86 | GABRG3 |
| 15q13.1 | 9266 | 25 | 1 | 2.61E−12 | 5.68E+01 | 29 | 3 | 2.15E−12 | 22.00 | OCA2 |
| 22q11.22 | 9572 | 9 | 2 | 6.96E−04 | 1.02E+01 | 29 | 2 | 2.86E−13 | 33.01 | LOC96610 |
| 22q11.22 | 359 | 12 | 38 | 8.84E−01 | 7.12E−01 | 76 | 39 | 4.43E−15 | 4.48 | ZNF280B |
| 22q11.22 | [symbol] | 28 | 5 | 1.68E−10 | 1.27E+01 | 77 | 5 | 6.12E−34 | 35.57 | ZNF280A |
| 22q11.22 | 1 | 18 | 4 | 1.06E−06 | 1.02E+01 | 71 | 4 | 6.77E−32 | 40.93 | PRAME |
| 22q11.22 | 3719 | 9 | 4 | 4.78E−03 | 5.09E−00 | 64 | 4 | 1.95E−28 | 36.81 | PRAME |
| | | | | | | | | | | LOC648691 |
| 22q11.22 | 5387 | 8 | 2 | 1.87E−03 | 9.05E−00 | 67 | 2 | 3.16E−32 | 77.17 | LOC648691 |
| 22q11.22 | 32636 | 5 | 2 | 3.18E−02 | 5.65E−00 | 56 | 2 | 1.13E−26 | 64.26 | GGTLC2 |
| | | | | | | | | | | POM121L1P |
| 22q11.22 | 18133 | 83 | 151 | 6.49E−02 | 1.25E−00 | 147 | 153 | 1.43E−11 | 2.22 | MIR650 |

The most significant CNVRs disrupting genes [symbol]
indicates data missing or illegible when filed

### Example II

### Screening Assays for Identifying Efficacious Therapeutics for the Treatment of Autism and ASD

The information herein above can be applied clinically to patients for diagnosing an increased susceptibility for developing autism or autism spectrum disorder and therapeutic intervention. A preferred embodiment of the invention comprises clinical application of the information described herein to a patient. Diagnostic compositions, including microarrays, and methods can be designed to identify the genetic alterations described herein in nucleic acids from a patient to assess susceptibility for developing autism or ASD. This can occur after a patient arrives in the clinic; the patient has blood drawn, and using the diagnostic methods described herein, a clinician can detect a CNV as described in Example I and set forth in Table II. The information obtained from the patient sample, which can optionally be amplified prior to assessment, will be used to diagnose a patient with an increased or decreased susceptibility for developing autism or ASD. Kits for performing the diagnostic method of the invention are also provided herein. Such kits comprise a microarray comprising nucleic acids containing at least one of the CNV/SNPs provided herein in and the necessary reagents for assessing the patient samples as described above.

The identity of autism/ASD involved genes and the patient results will indicate which variants are present, and will identify those that possess an altered risk for developing ASD. The information provided herein allows for therapeutic intervention at earlier times in disease progression than previously possible. Also as described herein above, the CNV containing genes described herein provide novel targets for the development of new therapeutic agents efficacious for the treatment of this neurological disease.

The information provided herein can also be employed in a test and treat approach. Although relatively common, the ASDs are still relatively under diagnosed, especially in rural areas where community pediatricians may not be as knowledgeable about the deluge of research that continues to define these disorders and their treatment. By and large, the mainstay of treatment for the ASDs remains behavioral therapy. There are many different types of behavioral therapies that specifically cater to the diverse phenotypic manifestations of the ASDs, and in all cases earlier intervention is correlated with better results. Therefore, early diagnosis of particular ASD subtypes is crucial to early behavioral intervention (and psychopharmacological management in extreme cases) and maximizing a child's potential and quality of life.

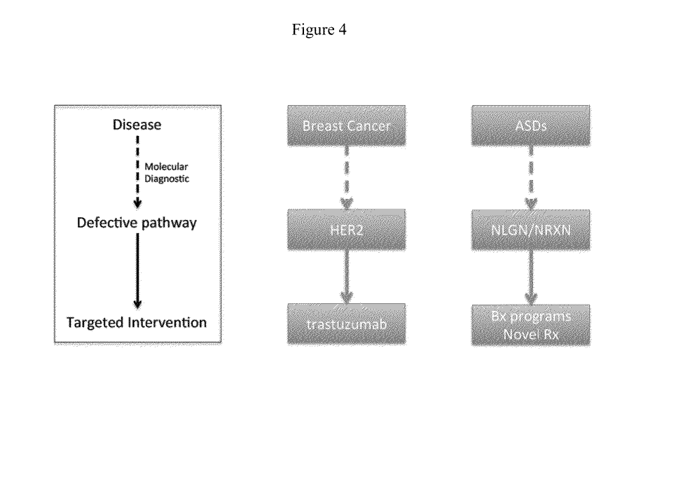

From a pharmacogenomics perspective using breast cancer as an analogy, trastuzumab, a monoclonal antibody that targets HER2 expressing cancer cells, has revolutionized the treatment for breast cancer, and it is perhaps the best-known example of a successful personalized therapeutic (FIG. 4). Before trastuzumab and other personalized therapeutics for cancer, patients would have to undergo relatively crude interventions such as radical mastectomy, radiation, and chemotherapy with low efficacy and severe undesirable side effects. When trastuzumab binds to defective HER2 proteins, it prevents the HER2 protein from causing cells in the breast to reproduce uncontrollably which increases the survival of breast cancer patients. Trastuzumab is only effective in HER2 expressing cancer cells so patients must undergo a genetic test to determine whether their cancer will respond to treatment. In fact, this test and treat model for targeted therapy is now the gold standard for breast cancer treatment.

Recently, next generation sequencing (NGS) was employed to study the genetic etiology of the ASD in sporadic families by analyzing the sequenced exomes of 20 parent-child trios, (O'Roak et al. 2011). These studies revealed four attractive candidate genes (FOXP1, GRIN2B, SCN1A and LAMC3) involved in neurotransmission which harbored functional de-novo mutations in sporadic families with ASDs. The notion that as few as 60 exomes could facilitate the identification of four rare plausible functional mutations underlying the ASDs suggests that as NGS of larger numbers of samples becomes more commonplace, the genomic landscape of rare mutations underlying ASDs will expand considerably to the benefit of clinicians and patients alike. Just as a better understanding of the molecular genetics of cancer cells has revolutionized the treatment of breast and other cancer treatments, improved resolution to identify rare mutations underlying ASDs will facilitate the development of molecular tests with better diagnostic yields that will be able to aid clinicians in diagnosing the ASDs and their particular genetic sub-types.

With better molecular diagnostics that dissect the sporadic genetic mutations underlying the ASDs, the personalized approach to treating patients' specific molecular defects in becomes a reality. This type of cutting-edge pharmacogenomics approach to the ASDs will facilitate the development of a test and treat model for drugs that target genetically defined responder populations just as trastuzumab does for HER2+ breast cancer patients (FIG. 4). Indeed SSRIs have already proved efficacious for rare cases of ASDs where the serotonin system is defective, and work is apace to define analogous treatments for ASDs due to malfunctioning NLGN/NRXN pathways, neuronal cell adhesion pathways, glutamatergic receptor pathways, and a host of other neurophysiological pathways and networks that have been shown to be defective in sporadic cases.

Having already implicated the GABAR-A pathway by CNV analysis as described above in Example 1, we have gone on to identify a host of drug candidates that act on this signalling pathway to potentially rescue underlying neurogenetic defects in patients with the ASDs (Tables 3 and 4). Topiramate is one such candidate that acts as an agonist at the GABAR-A pathway. Just as we did for the GABAR-A pathway itself in example 1, we defined the first-degree interactome of topiramate itself, and a found that ASD cases are significantly enriched for pathway defects in cases when compared with neurologically normal controls. About 20.7% of cases harbor genetic pathway defects vs 8.3% of controls, a statistically significant difference (P<=1.5×10⁻⁴⁴ OR=2.9) that supports our hypothesis that topiramate itself may be effective in treating patients with ASDs that harbor genetic defects in the GABAR-A pathway (FIG. 4).

The rational approach to personalized drug design as described herein should both restore normal neurophysiology in patients with ASDs by rescuing specific disrupted genetic pathways and avoid exposing them to drugs that will precipitate adverse side effects. Given the immense clinical and genetic heterogeneity of the ASDs, early tailored psychopharmacgenomic intervention as we have outlined here in combination with comprehensive behavioral programs should improve the prognosis and the outlook for patients that suffer from these burdensome diseases.

TABLE 3

| Active Ingredient | Phase | Description | Indication | MOA |
|---|---|---|---|---|
| | II | BL-1020 is an orally available small | Schizophrenia | Dopamine Receptor |
| | | molecule that acts as a dopamine receptor | | Antagonist; Gamma- |
| | | antagonist and GABA A receptor agonist. | | Aminobutyric Acid Type A |
| | | It is a novel antipsychotic agent, which | | (GABAA) Receptor Agonist |
| | | blocks hyperactivity of the | | |
| | | neurotransmitter dopamine, | | |
| | | simultaneously raising the levels of the | | |
| | | neurotransmitter GABA. BL-1020 is | | |
| | | being developed as an oral formulation | | |
| | | for the treatment of schizophrenia. | | |
| Amobarbital | M | Amylobarbitone belongs to barbiturates | Insomnia | Gamma-Aminobutyric Acid |
| | | that act to increase the actions of GABA | | Type A (GABAA) Receptor |
| | | which cause decreased nerve activity in | | Agonist |
| | | the brain it is developed to produce | | |
| | | sleepiness. It is indicated in the treatment | | |
| | | of insomnia and available as tablets | | |
| | | (10 mg, 25 mg, 50 mg). | | |
| butabarbital sodium | M | Butisol Sodium contains butabarbital | Insomnia | Gamma-Aminobutyric Acid |
| | | sodium as active ingredient. Butabarbital | | Type A (GABAA) Receptor |
| | | slows the brain and makes the person | | Agonist |
| | | calm and sleepy. Butisol Sodium is | | |
| | | indicated as a daytime sedative when mild | | |
| | | sedation is required for relief of | | |
| | | symptoms of anxiety or tension resulting | | |
| | | from emotional, physical, or situational | | |
| | | stress. It is available as tablets (30 mg, | | |
| | | 50 mg) and elixir (30 mg/5 ml) for oral | | |
| | | administration. Note: This product is | | |
| | | added upon the acquisition of MedPointe | | |
| | | Healthcare Inc. | | |
| Clomethiazole | M | Heminevrin contains clomethiazole, a | Insomnia | Gamma-Aminobutyric Acid |
| | | sedative and hypnotic that acts through | | Type A (GABAA) Receptor |
| | | the GABA receptor as an agonist and | | Agonist |
| | | enhances the neurotransmitter GABA. | | |
| | | Heminevrin is indicated for the treatment | | |
| | | of insomnia and is available in the form | | |
| | | of capsules (300 mg). | | |
| cyclobarbital calcium | M | Cyclobarbitone contains cyclobarbital | Epilepsy | Gamma-Aminobutyric Acid |
| | | calcium, a hypnotic and sedative acts on | | Type A (GABAA) Receptor |
| | | GABA-A receptors, increasing synaptic | | Agonist |
| | | inhibition which controls CNS arousal. It | | |
| | | is indicated for the treatment of epilepsy | | |
| | | and is available as tablets (50 mg, 100 mg, | | |
| | | 200 mg). | | |
| eszopiclone* | M | Eszopiclone, a nonbenzodiazepine | Insomnia | Gamma-Aminobutyric Acid |
| | | hypnotic agent is a pyrrolopyrazine | | Type A (GABAA) Receptor |
| | | derivative of the cyclopyrrolone class, | | Agonist |
| | | acts by interacting with GABA receptor | | |
| | | complexes at binding domains located | | |
| | | close to or allosterically coupled to | | |
| | | benzodiazepine receptors. S-Clon is used | | |
| | | for the treatment of insomnia and is | | |
| | | available as tablets. | | |
| etifoxine hydrochloride | M | Stresam contains etifoxine hydrochloride | Anxiety | Gamma-Aminobutyric Acid |
| | | a non benzodiazepine anxiolytic agent | | Type A (GABAA) Receptor |
| | | which acts by potentiating GABA (A) | | Agonist |
| | | receptor function by a direct allosteric | | |
| | | effect and by an indirect mechanism | | |
| | | involving the activation of peripheral | | |
| | | benzodiazepine receptors (PBRs). | | |
| | | Stresam is indicated for the treatment of | | |
| | | anxiety and is available as capsules | | |
| | | (50 mg). | | |
| Meprobamate | M | Meprobamate is a propanediol anxiolytic | Anxiety | Gamma-Aminobutyric Acid |
| | | agent which acts on the central nervous | | Type A (GABAA) Receptor |
| | | system causing muscle relaxation. | | Agonist |
| | | Meprobamate is indicated for the | | |
| | | treatment of anxiety and is available as | | |
| | | tablets (200 mg, 400 mg). | | |
| Moxidectin | III | Moxidectin is a potent broad-spectrum | Infection | Gamma-Aminobutyric Acid |
| | | endectocide of the macrocyclic lactone | (Onchocerciasis) | Type A (GABAA) Receptor |
| | | (macrolide) antimicrobial class. The | | Agonist |
| | | macrocyclic lactones consist of two | | |
| | | closely related chemical groups, | | |
| | | avermectins and milbemycins. | | |
| | | Moxidectin is being developed as oral | | |
| | | tablets for the treatment of onchocerciasis | | |
| | | (River Blindness). Note: This product is | | |
| | | added upon the acquisition of Wyeth. | | |
| Pagoclone | III | Pagoclone, a novel member of the | Stuttering | Gamma-Aminobutyric Acid |
| | | cyclopyrrolone class of compounds, | | Type A (GABAA) Receptor |
| | | which acts as a gamma amino butyric acid | | Agonist |
| | | (GABA) receptor modulator. It increases | | |
| | | the action of GABA, thus reducing excess | | |
| | | neuronal activity. IP456 is being | | |
| | | developed as an oral formulation for the | | |
| | | treatment of persistent developmental | | |
| | | stuttering. Note: This product is added | | |
| | | upon acquisition of Indevus | | |
| | | Pharmaceuticals Inc. | | |
| Phenobarbital | M | Phenobarbital is a barbiturate which acts | Epilepsy | Gamma-Aminobutyric Acid |
| | | on GABA A receptors by increasing | | Type A (GABAA) Receptor |
| | | synaptic inhibition that results in | | Agonist |
| | | elevating seizure threshold and reducing | | |
| | | the spread of seizure activity from a | | |
| | | seizure focus. Phenobarbital is indicated | | |
| | | for the treatment of epilepsy. It is | | |
| | | available as tablets (15 mg, 60 mg, 100 mg) | | |
| | | and injection (200 mg/ml). | | |
| Phenobarbital | M | Phenobarbitone is a barbituric acid | Insomnia | Gamma-Aminobutyric Acid |
| | | derivative, a nonselective central nervous | | Type A (GABAA) Receptor |
| | | system depressant which acts on GABAA | | Agonist |
| | | receptors, increasing synaptic inhibition. | | |
| | | This has the effect of elevating seizure | | |
| | | threshold and reducing the spread of | | |
| | | seizure activity from a seizure focus. | | |
| | | Phenobarbitone may also inhibit calcium | | |
| | | channels, resulting in a decrease in | | |
| | | excitatory transmitter release. The | | |
| | | sedative-hypnotic effects of phenobarbital | | |
| | | are likely the result of its effect on the | | |
| | | polysynaptic midbrain reticular | | |
| | | formation, which controls CNS arousal. | | |
| | | Phenobarbitone is indicated for the | | |
| | | treatment of insomnia and is available as | | |
| | | tablets (15 mg, 30 mg, 60 mg, and 100 mg). | | |
| phenobarbital sodium | M | Phenobarbital sodium is a longest-acting | Epilepsy | Gamma-Aminobutyric Acid |
| | | barbiturate with anticonvulsant and | | Type A (GABAA) Receptor |
| | | sedative-hypnotic properties. It acts on | | Agonist |
| | | GABAA receptors, increasing synaptic | | |
| | | inhibition which results in elevating | | |
| | | seizure threshold and reducing the spread | | |
| | | of seizure activity from a seizure focus. | | |
| | | Phenobarbital may also inhibit calcium | | |
| | | channels, resulting in a decrease in | | |
| | | excitatory transmitter release. It is | | |
| | | indicated for the treatment of epilepsy and | | |
| | | available as intramuscular and | | |
| | | intravenous injection (65 mg/ml and | | |
| | | 130 mg/ml). | | |
| Phenprobamate | M | Phenprobamate is a centrally acting | Spasm | Gamma-Aminobutyric Acid |
| | | skeletal muscle relaxant. It acts by | | Type A (GABAA) Receptor |
| | | interrupting neuronal communication in | | Agonist |
| | | the reticular formation and spinal cord, | | |
| | | causing sedation and altered perception of | | |
| | | pain. Phenprobamate is indicated for the | | |
| | | treatment of musculoskeletal and muscle | | |
| | | spasms and is available as tablets (0.2 g). | | |
| Primidone | M | Primidone is an anticonvulsant agent that | Epilepsy | Gamma-Aminobutyric Acid |
| | | is structurally related to barbiturates. It is | | Type A (GABAA) Receptor |
| | | a gamma-aminobutyric acid type (GABA) | | Agonist |
| | | receptor agonist that increases the | | |
| | | synaptic inhibition, elevating seizure | | |
| | | threshold and reducing the spread of | | |
| | | seizure activity from a seizure focus. | | |
| | | Primidone is indicated alone or | | |
| | | concomitantly with other anticonvulsants | | |
| | | for the control of grand mal, psychomotor | | |
| | | and focal epileptic seizures. It is available | | |
| | | as tablets (50 mg and 250 mg). | | |
| Topiramate | M | Adco-Topiramate contains topiramate, | Epilepsy | Alpha-Amino-3-Hydroxy-5- |
| | | a sulfamate-substituted | | Methyl-4- |
| | | monosaccharide and an anticonvulsant | | Isoxazolepropionic Acid |
| | | agent. It blocks the voltage dependent | | (AMPA) Receptor |
| | | sodium channels, augments the activity | | Antagonist; Gamma- |
| | | of the neurotransmitter gamma amino | | Aminobutyric Acid Type A |
| | | butyrate at some subtypes of the | | (GABAA) Receptor |
| | | GABA-A receptor, antagonizes the | | Agonist; Sodium Channel |
| | | AMPA/kainate subtype of the | | Blocker |
| | | glutamate receptor and inhibits the | | |
| | | carbonic anhydrase enzyme, | | |
| | | particularly isozymes II and IV. Adco- | | |
| | | Topiramate is indicated for the | | |
| | | treatment of partial onset seizures, | | |
| | | primary generalized tonic clonic | | |
| | | seizures and seizures associated with | | |
| | | lennox-gastaut syndrome. It is available | | |
| | | as tablets (25 mg, 50 mg, 100 mg, 200 mg). | | |
| zaleplon* | M | Zaleplon is a short-acting, non- | Insomnia | Gamma-Aminobutyric Acid |
| | | benzodiazepine sedative-hypnotic. It acts | | Type A (GABAA) Receptor |
| | | as an agonist at type 1 benzodiazepine | | Agonist |
| | | (BZ1 or omega1) receptors on the | | |
| | | GABA-A/chloride-ion channel complex | | |
| | | within the CNS. Zaleplon is indicated for | | |
| | | the treatment of insomnia and is available | | |
| | | as capsules (5 mg and 10 mg). | | |
| zolpidem* | M | Zolpidem is a non-benzodiazepine | Insomnia | Gamma-Aminobutyric Acid |
| | | hypnotic of the imidazopyridine class. It | | Type A (GABAA) Receptor |
| | | acts by increasing the activity of GABA, | | Agonist |
| | | there by reducing the functioning of | | |
| | | certain areas of the brain. It results in | | |
| | | sleepiness, decrease in anxiety and | | |
| | | relaxation of muscles. Zolpidem is | | |
| | | indicated for the treatment of insomnia | | |
| | | and is available as film coated tablets | | |
| | | (5 mg, 10 mg). | | |
| zolpidem tartrate* | M | Zolfresh contains zolpidem tartrate, a | Insomnia | Gamma-Aminobutyric Acid |
| | | nonbenzodiazepine sedative-hypnotic, | | Type A (GABAA) Receptor |
| | | which belongs to the imidazopyridine | | Agonist |
| | | class of drugs. It is a gamma-amino-n- | | |
| | | butyric acid type A (GABA-A) receptor | | |
| | | agonist which works by binding | | |
| | | preferentially to the omega-1 (BZ-1) | | |
| | | receptor subtype of the GABAA receptor | | |
| | | complex. Zolfresh is indicated for the | | |
| | | short-term treatment of insomnia and is | | |
| | | available as film coated tablets (5 mg, | | |
| | | 10 mg). | | |
| zopiclone* | M | Zopimed contains zopiclone, a | | Gamma-Aminobutyric Acid |
| | | nonbenzodiazepine hypnotic of the | | Type A (GABAA) Receptor |
| | | pyrazolopyrimidine class. It binds | | Agonist |
| | | selectively to the brain alpha subunit of | | |
| | | the gamma aminobutyric acid (GABA A) | | |
| | | omega 1 receptor and exerts its action by | | |
| | | modulation of the GABA benzodiazepine | | |
| | | receptor chloride channel macromolecular | | |
| | | complex. Zopimed is indicated for the | | |
| | | short-term treatment of insomnia in adults | | |
| | | and is available as tablets (7.5 mg). | | |

GABA-A agonist drugs
*structurally not like benzodiazepine, but have similar effects.

TABLE 4

| | Active | | | |
|---|---|---|---|---|
| Product | Ingredient | Phase | Indication | MOA |
| NGD912 | — | F | Anxiety | Gamma-Aminobutyric Acid (GABA) Receptor Agonist |
| NGD913 | — | F | Anxiety | Gamma-Aminobutyric Acid (GABA) Receptor Agonist |
| Gaboxadol | THIP | D | Insomnia | GABA-A recceptor agonist |
| CPP109 | Vigabatrin | I | Drug Abuse (Addiction | Gamma-Aminobutyric Acid Transaminase (GABA-T) Inhibitor |
| | | | on both Cocaine and | |
| | | | Alcohol) | |
| CPP115 | — | I | Cocaine Addiction | Gamma-Aminobutyric Acid Transaminase (GABA-T) Inhibitor |
| SUN09 | — | I | Neurological Disorders | Gamma-Aminobutyric Acid Type B (GABAB) Receptor Agonist |
| | | | (Muscle Spasticity) | |
| AGI006 | Arbaclofen | II | Indigestion (Functional | Gamma-Aminobutyric Acid Type B (GABAB) Receptor Agonist |
| | | | Dyspepsia) | |
| DP-VPA | — | II | Epilepsy | Gamma-Aminobutyric Acid Transaminase (GABA-T) Inhibitor |
| Xyrem | sodium | II | Schizophrenia | Gamma-Aminobutyric Acid Type B (GABAB) Receptor Agonist |
| | oxybate | | | |
| STX209 | Arbaclofen | III | Fragile X syndrome | |
| IP456 | Pagoclone | III | Stuttering | Gamma-Aminobutyric Acid Type A (GABAA) Receptor Agonist |
| XP19986 | arbaclofen | III | Neurological disorders | Gamma-Aminobutyric Acid Type B (GABAB) Receptor Agonist |
| | placarbil | | | |
| | Baclofen | M | Spasm | Gamma-Aminobutyric Acid Type B (GABAB) Receptor Agonist |
| | Vigabatrin | M | Epilepsy | Gamma-Aminobutyric Acid Transaminase (GABA-T) Inhibitor |
| Xyrem | sodium | M | Marcolepsy | Gamma-Aminobutyric Acid Type B (GABAB) Receptor Agonist |
| | oxybate | | | |
| Lyrica | Pregabalin | M | Anxiety, | GABA analog |
| | | | epilepsy, Neuralgia, | |
| | | | Diabetic | |
| | Gabapentin | M | Epilepsy | GABA analog |
| Heminevrin | clomethiazole | M | Insomenia in Europe | Gamma-Aminobutyric Acid Type A (GABAA) Receptor Agonist |
| Gabitril | tiagabine | M | Epilepsy | GABA reuptake inhibitor |
| | hydrochloride | | | |

### Example III

### The Impact of the Metabotropic Glutamate Receptor and Other Gene Family Interaction Networks on Autism

Despite being highly heritable, the vast majority of family studies suggest that the ASDs do not segregate as a simple Mendelian disorder, but rather display clinical and genetic heterogeneity consistent with a complex trait [13]. Indeed, recent studies estimate that the ASDs may comprise up to 400 distinct genetic and genomic disorders that phenotypically converge [14,15]. Common variants such as single-nucleotide polymorphisms seem to contribute to ASD susceptibility, but, taken individually, their effects appear to be small [16]. However, there is increasing evidence that the ASDs can arise from rare or “private” highly penetrant mutations that segregate in families but are less generalizable to the general population[17-19]. Many genes implicated thus far-which are involved in chromatin remodeling, metabolism, mRNA translation, and synaptic function-seem to converge in common pathways or genetic networks affecting neuronal and synaptic homeostasis [16].

Such remarkable phenotypic and genotypic heterogeneity when coupled with the private nature of mutations in the ASDs has hindered identification of new genetic risk factors with therapeutic potential. However, it is noteworthy that many of the rare gene defects implicated in the ASDs belong to gene families. For instance, rare defects impacting multiple members of both the post-synaptic neuroligin (NLGN) gene family [20] as well as their pre-synaptic neurexin (NRXN) molecular interacting partners [21,22] have long been reported in patients with ASDs. Additionally, a number of other defective gene families with important functional roles have subsequently been well-characterized including ubiquitin (UBEA) conjugation[23], gamma-aminobutyric acid (GABA) receptor signaling [24-27] and cadherin/protocadherin (CDH) cell junction proteins [28] in the brain. Furthermore, multiple defects in voltage gated calcium channels (CACNA) have been found in schizophrenia [29], and a defective network of metabotropic glutamate (GRM) receptor signaling was found in both ADHD [30] and schizophrenia [31-36], two neuropsychiatric disorders that are highly coincident with the ASDs. Also, the vast majority of significant defective genes identified from recent whole exome sequences belongs to gene families [17-19].

Many studies have found defective genetic networks in the ASDs, [21,23,37-40] (see [16] for review), and we complement these in this work by uncovering new networks and implicating specific defective gene families that may be enriched for novel potential therapeutic targets. Drug binding sites on proteins usually exist out of functional necessity [33], and gene families derive from gene duplication events that present additional binding sites for a given drug to exert its effects. Most successful drugs achieve their activity by competing for a binding site on a protein with an endogenous small molecule [41], therefore, many successful pharmacologic gene targets are within large gene families. Indeed, nearly half of the pharmacologic gene targets fall into just six gene families: G-protein-coupled receptors (GPCRs), serine/threonine and tyrosine protein kinases, zinc metallopeptidases, serine proteases, nuclear hormone receptors and phosphodiesterases [41]. Moreover, many large gene families are localized to pre and post synaptic neuronal terminals to coordinate the highly complex and evolutionarily conserved process of neurotransmission[42], which is thought to be compromised to varying degrees in the autistic brain [43]. Therefore we hypothesize that we may select more efficacious drug targets for the ASDs by enriching for defective interaction networks defined by gene families.

The following materials and methods are provided to facilitate the practice of Example III.

### Ethics Statement

The research presented here has been approved by the Children's Hospital of Philadelphia IRB (CHOP IRB#: IRB 06-004886). Some patients and their families were recruited through CHOP outreach clinics. Written informed consent was obtained from the participants or their parents using IRB approved consent forms prior to enrollment in the project. There was no discrimination against individuals or families who chose not to participate in the study. All data were analyzed anonymously and all clinical investigations were conducted according to the principles expressed in the Declaration of Helsinki.

### Samples, Genotyping, and Ethnicity Inference

Samples were selected from DNA collected as part of the Center for Applied Genomics (CAG) biorepository, from samples that originated at the Children's Hospital of Philadelphia. All children had a community diagnosis of ASD (n=539). This cohort included any children with ASD, and was unfiltered for presence of a comorbid genetic syndrome.

### Clinical Characterization:

A physician blinded to mGluR status conducted chart review for all patients with mGluR network CNV's (n=62), 100 patients without mGluR CNV's, and all patients in the 22q sample (n=78). Patients were excluded if there was insufficient documentation of a community diagnosis of ASD. The validity of this diagnosis was not assessed as part of the present study. Patients were also excluded if they did not have at least one comprehensive history and physical documented by a physician. Comorbid medical conditions, clinical genetic testing and imaging data were reviewed. Children were categorized as having “Syndromic ASD” if they had ASD plus a structural defect or medical condition that occurs in less than 1% of the general population, and/or diagnosis of a genetic syndrome based on clinical testing. Genetic abnormalities predicted to be benign or ‘variant’ findings on clinical array were not categorized as “syndromic ASD” unless they met criteria based on the aforementioned clinical abnormalities. Two patients without documented structural abnormalities (but significant developmental delay) who had not received clinical genetic testing were categorized as “Syndromic ASD” based on the presence of large deletions on research arrays.

The majority of cases (5,049 of 6,742) and all controls (12,544) were genotyped with genome wide coverage using the Infinium II platform across various iterations of the HumanHap BeadChip with 550K, 610K, 660K, and 1M markers by the Center for Applied Genomics at The Children's Hospital of Philadelphia (CHOP). There were 1,693 cases genotyped by the AGP consortium. All cases and approximately 50% of controls were re-used from previously published large ASD studies [21,23,28,44]. All cases were diagnosed by ADI-R/ADOS and fulfilled standard criteria for autism spectrum disorders. Duplicates samples were removed by selecting unique samples with the best quality (based on genotyping statistics used to QC samples) from clusters defined by single linkage clustering of all pairs of samples with high pairwise identity by state measures (IBS>=0.9) across 140K non-correlated SNPs. Ethnicity of samples was inferred by a supervised k-means classification (k=3) of the first 10 eigenvectors estimated by principal component analysis across the same subset of 140K non-correlated SNPs. We used HapMap 3 [45] and the Human Genome Diversity Panel [46] samples with known continental ancestry to train the k-means classifier implemented by the R Language for Statistical Computing [65].

### CNV Inference and Association

We called CNVs with the PennCNV algorithm [66], which combines multiple values, including genotyping fluorescence intensity (Log R Ratio), population frequency of SNP minor alleles (B Allele Frequency), and SNP spacing into a hidden Markov model. The term ‘CNV’ represents individual CNV calls, whereas ‘CNVR’ refers to population-level variation shared across subjects. Quality control thresholds for sample inclusion in CNV analysis included a high call rate (call rate>=95%) across SNPs, low standard deviation of normalized intensity (SD<=0.3), low absolute genomic wave artifacts (|GCWF|<=0.02), and low numbers of CNVs called (#CNVs<=100). Genome wide differences in CNV burden, defined as the average span of CNVs, between cases and controls and estimates of significance were computed using PLINK [67]. CNVRs were defined based on the genomic boundaries of individual CNVs, and the significance of the difference in CNVR frequency between cases and controls was evaluated at each CNVR using Fisher's exact test.

### Gene Family Interaction Networks (GFINs) Definition and Association

We extended our previous work from Example I to rank all gene family interaction networks (GFINs) by a permutation test. Specifically, we defined a GFIN as the directed second-degree gene interaction network defined by a family of genes. We found 2,611 gene families with at least two members based on official HUGO [48] gene nomenclature, and generated 1,732 GFINs using merged human interactome data from three different yeast two hybrid generated datasets [49-51] accessed through the Human Interactome Database [68]. We calculated an enrichment of cumulative network enrichment in a method previously described [30] for 1,557 GFINs with defined CNVs.

For each GFIN, we quantified its enrichment by a permutation test of 1000 second-degree gene interaction networks derived from a random set of N genes, where N is the number of members of a given gene family. Because the CNVs we are focused on are so rare, we are underpowered to achieve significance py permutation testing after correcting for multiple GFIN tests. However, we report all GFINs that are nominally significant.

### CNV Quality Control:

Samples with SNP arrays of poor quality were excluded from CNV calling, since typically the proportion of false positives increases considerably for these samples. Only those samples where the genotyping call rate>98%, standard deviation of LRR (LRR_sd)<0.35, GC-wave factor (GCWF) is between −0.2 and 0.2, and total number of CNV calls for the sample <100. CNV's were visually valiated based on ParseCNV criteria (Glessner et al Plos One, 2013).

### CNV Annotation

For syndromic ASD regions, genomic coordinates were those described by Betancur (2011 Brain Res). The GRM/mGluR network generated by Cytoscape from the Human Interactome database was described by Elia et al. (2012 Nat Genet) using UCSC Genome Browser definitions for gene coordinates. CNV calls were analyzed for overlap to known syndromic regions and GRM network genes. All syndromic aberrations detected by clinical cytogenetic laboratory testing were confirmed on corresponding SNP arrays.

### Statistical Analysis

Group comparisons were made using Fisher's Exact Test and Chi Square sample distribution as previously described.

### Experimental Validation of CNVs

Significant CNVRs that we identified were validated using commercially available qPCR Taqman probes run on the ABI GeneAmp 9700 system from Life Technology. Data File 1 lists 251 reactions that we tested using 121 different genomic probes across 85 different samples for which DNA was available. For deletions, our sensitivity=0.65, specificity=1.00, NPV=1.00, and PPV=0.88. For duplications, our sensitivity=0.68, specificity=0.99, NPV=0.94, and PPV=0.91.

### Results

In the present example we describe the results from a large genome-wide association study (GWAS) of structural variants that disrupt gene family protein interaction networks in patients with autism. We find multiple defective networks in the ASDs, most notably rare copy number variants (CNVs) in the metabotropic glutamate receptor (mGluR) signaling pathway in up to 6% of patients with the ASDs (as described above in Example I). Defective mGluR signaling was found in both ADHD [30] and schizophrenia [31-36], two common neuropsychiatric disorders that are highly coincident with the ASDs. Furthermore, we find other attractive candidates such the MAX Dimerization Protein (MXD) network that is implicated in cancer, and a Calmodulin 1 (CALM1) gene interaction network that is active in neuronal tissues. The numerous defective gene family interactions we find to underlie autism present many novel translational opportunities for the generation of more effective therapeutic interventions.

To identify and comprehensively characterize defective genetic networks underlying the ASDs, we performed a large-scale genome association study for copy number variation (CNVs) enriched in patients with autism. By combining the affected cases from previously published large ASD studies [21, 23, 28, 44] with more recently recruited cases from the Children's Hospital of Philadelphia, we executed one of the largest searches for rare pathogenic CNVs in ASDs to-date. In sum, 6,742 genotyped samples from patients with the ASDs were compared to those from 12,544 neurologically normal controls recruited at The Children's Hospital of Philadelphia (CHOP). These cases were each screened by neurodevelopmental specialists to exclude patients with known syndromic causes for autism. Genotyping was performed at CHOP for the vast majority of the ASD cases as well as all the controls. After cleaning the data to remove sample duplicates and performing standard QC for CNVs, we first inferred the continental ancestry of 5,627 affected cases and 9,644 disease free controls using a training set defined by populations from HapMap 3 [45] and the Human Genome Diversity Panel [46] (Table 5). Using this QC criteria, we estimated that the sensitivity and specificity of calling CNVs is approximately 70% and 100%, respectively, across 121 different genomic regions assayed by PCR (see methods). Across all ethnicities, there was an increased burden of CNVs in cases vs controls, a statistically significantly difference (P<=0.001) in the larger European (63.3 vs 54.5 Kb respectively) and African (70.4 vs 48.0 Kb respectively) derived populations.

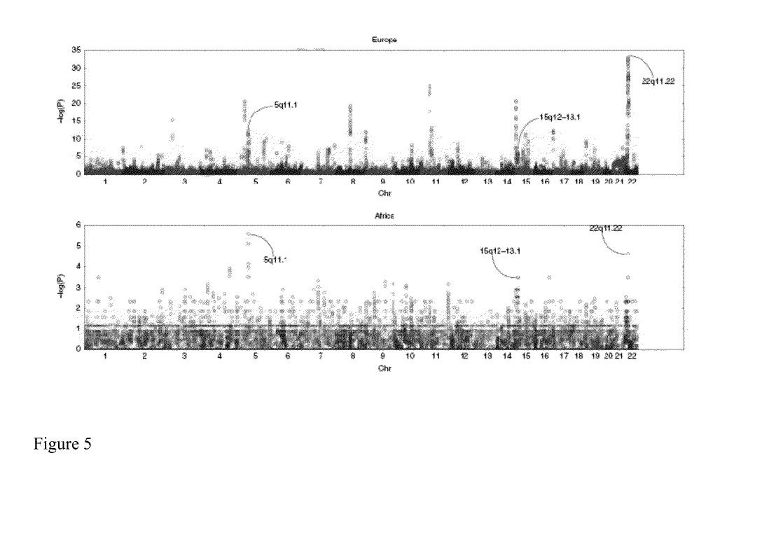

We then searched for pan-ethnic CNV regions (CNVRs) discovered in the European-derived dataset (4,602 cases vs 4,722 controls; P<=0.0001) and replicated in an independent ASD dataset of African ancestry (312 cases vs 4,169 controls; P<=0.001) with subsequent measurement of overall significance across the entire multi-ethnic discovery cohort (5,627 cases vs 9,644 controls) for maximal power (FIG. 5, Table 6). Based on these selection criteria, two large well-known ASD risk loci emerged that harbored multiple duplications in the Prader Willi/Angelman syndrome (15q11-13) critical region and multiple deletions were detected in the DiGeorge syndrome (22q11) critical region, albeit notably smaller than the 22q11 deletion syndrome. A third locus harboring deletions in poly ADP-ribose polymerase family 8 (PARP8) on chromosome 5₈11 was also discovered. PARP8 was previously identified as associated with the ASDs in a Dutch population[47], but it has not previously been described for its pan ethnic distribution across European-derived and African-derived populations.

We examined the genetic interaction networks derived from gene families with members localized to the the Prader Willi/Angelman syndrome (15q11-13) critical region, the DiGeorge syndrome (22q11) critical region, and the novel PARP8 (5q11) region using a method previously applied to ADHD [30]; however, hardly any of the most significant genes harboring significant CNVRs clustered within gene families. Consequently, we broadened our search for gene family interaction networks (GFINs) and searched the entire genome for GFINs with CNVs enriched in autism. For every gene family, we defined a GFIN as the genetic interaction network spawned by its multiple duplicated members. We used standard HUGO [48] gene names to define 1,732 GFINs across which we searched for enrichment of network defects associated with the ASDs. However, because there is an a-priori excess of CNV burden in ASD cases over disease free controls (Table 5), larger GFINs are expected to display significant enrichment of case defects by virtue solely of their increased size and complexity. Therefore, for each GFIN, we used a network permutation test of case enrichment across 1,000 random sets of networked genes to control for the GFIN size and complexity. With this approach, we robustly identified network defects associated with the ASDs by minimizing statistical artifact derived from any a priori excessive CNV burden in cases over controls, as well as other unknown biases that may be inherent in the human interactome data [49-51] that we mined.

TABLE 5
Distribution of cases, controls, and CNV coverage across
estimated continental ancestry. For groups of cases and
controls across estimated ancestries, the table lists the
numbers of subjects that passed quality control and their
group-wise CNV burden, defined as the average span of
CNVs in Kb for each group.

| Continental Ancestry | Case | Control | Total |
|---|---|---|---|
| Europe | | | |
| Number of samples | 4,602 | 4,722 | 9,324 |
| *CNV burden (Kb) | 63.3 | 54.5 | |
| Africa | | | |
| Number of samples | 312 | 4,169 | 4,481 |
| *CNV burden (Kb) | 70.4 | 48.0 | |
| America | | | |
| Number of samples | 485 | 276 | 761 |
| CNV burden (Kb) | 59.1 | 58.4 | |
| Asia | | | |
| Number of samples | 201 | 350 | 551 |
| CNV burden (Kb) | 56.1 | 54.1 | |
| Other | | | |
| Number of samples | 27 | 127 | 154 |
| CNV burden (Kb) | 51.5 | 49.4 | |
| All Ethnicities | | | |
| Number of samples | 5,627 | 9,644 | 15,271 |
| *CNV burden (Kb) | 63.0 | 51.7 | |

*Statistically significant (P <= 0.01) differences in CNV burden are marked with an asterix (*).

TABLE 6
Copy number variable regions (CNVRs) distinguishing cases from controls significant across both
European-derived populations (P <= 0.0001) and African-derived populations (P <= 0.001). For
each CNVR, the table lists the type (del or dup), the closest gene impacted, the chromosomal band,
the approximate size of the defect (Kb), the number of contributing SNPs, the numbers of affected
cases and controls, as well as P value and odds ratio (OR) from Fisher's exact test for across
all populations, and subsets of European-derived and African-derived populations.

| | | | Size | # | # | # | All | | Europe | | Africa | |
|---|---|---|---|---|---|---|---|---|---|---|---|---|
| CNVR | Genes | bands | (Kb) | SNP | Case | Ctrl | P | OR | P | OR | P | [symbol] |
| Del | ZNF280B | 22q11.22 | 53.4 | 13 | 130 | 0 | 2.56E−57 | Inf | 1.94E−33 | Inf | 3.34E−04 | [symbol] |
| Del | *PARP8 | 5q11.1 | 47.7 | 8 | 70 | 8 | 2.76E−22 | 15.1 | 3.84E−13 | 12.0 | 2.69E−06 | [symbol] |
| Dup | *GABRB3 | 15q12 | 49.0 | 20 | 28 | 0 | 7.60E−13 | Inf | 1.50E−06 | Inf | 3.34E−04 | [symbol] |
| Dup | *GABRG3 | 15q12 | 135.3 | 13 | 27 | 1 | 3.72E−11 | Inf | 1.60E−05 | 19.5 | 3.34E−04 | [symbol] |
| Dup | *HERC2 | 15q13.1 | 84.4 | 2 | 24 | 0 | 4.12E−11 | Inf | 6.17E−06 | Inf | 3.34E−04 | [symbol] |

*Genes with an asterix (*) harbor CNVRs that disrupt their exons of directly, while those without the asterix are located in the genomic region around the intergenic CNVRs. [symbol]
indicates data missing or illegible when filed

TABLE 7
Top gene family interaction networks discovered. The table shows significant gene family
interaction networks (GFINs) by network permutation testing (Pₚₑᵣₘ <= 0.05) enriched
for CNV defects across at least 5% of cases. The table lists the name and size of gene
family tested, the number and frequency of network genes enriched in the second degree
gene interaction network, the number and frequency of cases harboring defects across the
network, the number and frequency of controls harboring defects across the network, the
significance of association by Fisher's exact test, the enrichment of CNV defects in
cases, and the significance of that enrichment by 1000 random network permutations.

| Gene Family | | Enriched Genes | | Cases | | Controls | | Gene Network Association | | |
|---|---|---|---|---|---|---|---|---|---|---|
| Name | Size | # | Freq | # | Freq | # | Freq | P_{fisher} | Enrichment | Pₚₑᵣₘ |
| BRF | 2 | 242/326 | 0.742 | 567 | 0.123 | 370 | 0.078 | 3.30E−13 | 1.65 | 0.040 |
| CCL | 24 | 108/144 | 0.75 | 231 | 0.05 | 129 | 0.027 | 5.62E−09 | 1.88 | 0.008 |
| CCNT | 2 | 183/254 | 0.72 | 613 | 0.133 | 381 | 0.081 | 1.10E−16 | 1.75 | 0.007 |
| ELAVL | 4 | 108/156 | 0.692 | 327 | 0.071 | 152 | 0.032 | 6.87E−18 | 2.3 | 0.043 |
| ERCC | 7 | 263/369 | 0.713 | 836 | 0.182 | 560 | 0.119 | 7.67E−18 | 1.65 | 0.035 |
| GRM | 8 | 124/181 | 0.685 | 265 | 0.058 | 153 | 0.032 | 2.40E−09 | 1.82 | 0.043 |
| GTF2H | 5 | 152/223 | 0.682 | 391 | 0.085 | 233 | 0.049 | 3.21E−12 | 1.79 | 0.049 |
| KIAA | 106 | 268/373 | 0.718 | 988 | 0.215 | 647 | 0.137 | 3.12E−23 | 1.72 | 0.045 |
| KPNA | 7 | 256/367 | 0.698 | 560 | 0.122 | 369 | 0.078 | 1.26E−12 | 1.63 | 0.028 |
| MXD | 3 | 52/64 | 0.813 | 366 | 0.08 | 156 | 0.033 | 3.83E−23 | 2.53 | 0.042 |
| POU5F | 2 | 94/130 | 0.723 | 293 | 0.064 | 131 | 0.028 | 2.96E−17 | 2.38 | 0.041 |
| RAD | 7 | 218/309 | 0.706 | 535 | 0.116 | 339 | 0.072 | 9.68E−14 | 1.7 | 0.042 |
| SAP | 4 | 111/150 | 0.74 | 274 | 0.06 | 151 | 0.032 | 9.61E−11 | 1.92 | 0.040 |
| SMAD | 8 | 845/1225 | 0.69 | 1782 | 0.387 | 1424 | 0.302 | 1.81E−18 | 1.46 | 0.039 |
| SMARCC | 2 | 106/147 | 0.721 | 239 | 0.052 | 131 | 0.028 | 1.22E−09 | 1.92 | 0.043 |
| SMC | 5 | 88/120 | 0.733 | 336 | 0.073 | 176 | 0.037 | 1.71E−14 | 2.03 | 0.034 |

TABLE 8
Most significant individual gene interaction networks ranked by permutation testing.
The table lists the name and gene family member tested, the number and frequency of
network genes enriched, the number and frequency of cases harboring defects, the number
and frequency of controls harboring defects, and the significance of association by
Fisher's exact test, the odds ratio of the effect size, and the significance of association
my random permutation of network while controlling for number of genes tested.

| Gene Family | Enriched Genes | | Cases | | Controls | | Gene Network Association | | |
|---|---|---|---|---|---|---|---|---|---|
| Member | # | Freq | # | Freq | # | Freq | P_{fisher} | Enrichment | Pₚₑᵣₘ |
| AKAP13 | 7/7 | 1.00 | 16 | 0.0035 | 1 | 0.0002 | 1.14E−04 | 16.43 | 0.012 |
| BAG1 | 7/7 | 1.00 | 15 | 0.0032 | 1 | 0.0002 | 2.18E−04 | 15.40 | 0.014 |
| CALM1 | 9/10 | 0.90 | 14 | 0.0030 | 1 | 0.0002 | 4.16E−04 | 14.37 | 0.002 |
| CASP6 | 16/17 | 0.94 | 46 | 0.0100 | 6 | 0.0013 | 2.96E−09 | 7.91 | 0.012 |
| GTF2H3 | 23/26 | 0.88 | 42 | 0.0091 | 8 | 0.0017 | 3.66E−07 | 5.41 | 0.009 |
| MAP3K5 | 11/12 | 0.92 | 34 | 0.0074 | 4 | 0.0008 | 2.02E−07 | 8.76 | 0.012 |
| NCOR1 | 9/10 | 0.90 | 26 | 0.0056 | 2 | 0.0004 | 1.11E−06 | 13.37 | 0.004 |
| PARP1 | 5/5 | 1.00 | 5 | 0.0011 | 0 | 0.0000 | 2.95E−02 | Inf | 0.012 |
| PTPN13 | 6/6 | 1.00 | 9 | 0.0019 | 0 | 0.0000 | 1.75E−03 | Inf | 0.007 |
| TCEA1 | 22/26 | 0.85 | 39 | 0.0084 | 7 | 0.0015 | 5.94E−07 | 5.74 | 0.009 |

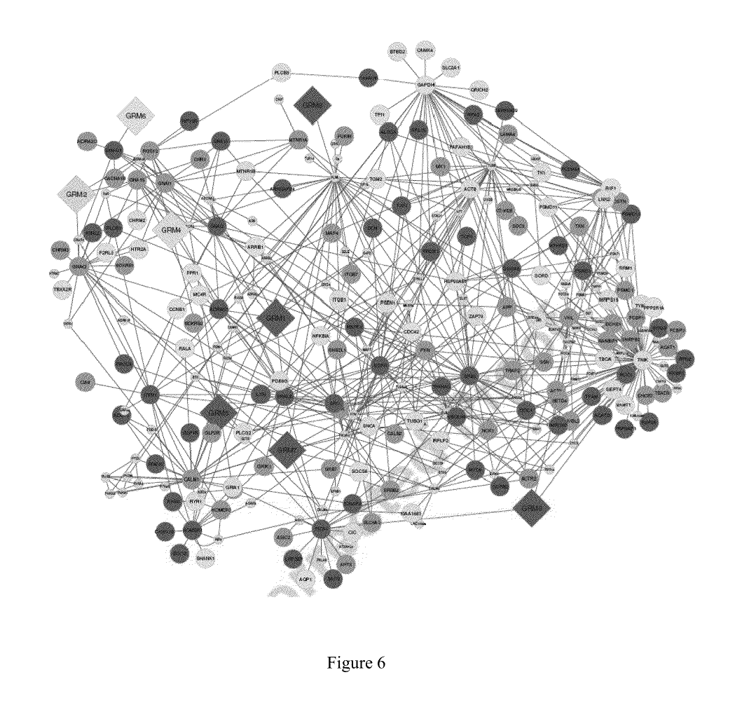

Out of 1,732 GFINs, we used the network permutation test to rank 1,557 GFINs with defined CNVs for enrichment of genetic defects in the ASDs. Among the top GFINs (Table 7) was the metabotropic glutamate receptor (mGluR) pathway defined by the GRM family of genes that impacts glutaminergic neurotransmission. The GRM family contains eight members, all of which were defined in the human interactome to cumulatively spawn a GFIN of 279 genes (FIG. 6). Across this GFIN for the GRM family of genes, we found CNV defects in 5.8% of European-derived ASD cases (265/4602) vs only 3% of ethnically matched controls (153/4722), a 1.8-fold enrichment of frequency (P_{Fisher}<=2.40E-09). By 1,000 random network permutations, we found this excess of enrichment across cases in the mGluR pathway to also be statistically significant (Pₚₑᵣₘ<=0.05). Additionally, 69.2% (124/181) of the informative genes within our mGluR network showed an excess of CNVs among cases. However, the component genes that harbor the most significant CNVRs contributing to this overall network significance reveal that the duplicated mGluR genes themselves (GRM1, GRM3, GRM4, GRM5, GRM6, GRM7, and GRM8) fail to achieve significance individually, although there is a trend for an excess of CNV defects across a specific subset of mGluR receptors (GRM1, GRM3, GRM5, GRM7, GRM8) that is unique to cases (data not shown).

Many large studies of CNVs implicate genes within the glutaminergic signaling pathway in the etiology of the ASDs [21,23,37-40], and SNP [52,53] and CNV duplications [54] of GRM8 have been reported in association with the ASDs before in humans. Moreover, a recent functional study demonstrated that in mouse models of tuberous sclerosis and fragile X, two different forms of syndromic autism, the autistic phenotype was ameliorated by modulation of GRM5 in opposite directions for each syndrome which suggests that GRM5 functional activity is central in defining the axis of synaptopathophysiology in syndromic autism [55]. Our GRM network findings implicate rare defects in mGluR signaling also contribute to the ASDs outside of fragile X and tuberous sclerosis, and we posit that functional mGluR synaptopathophysiology may be initiated from many dozens if not hundreds of defective genes within the mGluR pathway that may account for as much as 6% of the endophenotypes of the ASDs (Table 7). Additionally, we recently demonstrated the importance of mGluRs in ADHD [30,56], a highly co-incident neuropsychiatric disorder within the autism spectrum. However, in contrast to ADHD where defects within the mGluR receptors themselves (GRMs) were among the most significant copy number defects contributing to the overall network significance, we found that in the ASDs defects of component GRMs contributed only modestly to the overall significance of the mGluR pathway. Nonetheless, the defects within GRM1, GRM3, GRM5, GRM7, and GRM8 that we identified as unique to cases and thus enriched are the same GRMs we identified as being pathogenic in ADHD and may impact glutaminergic signaling.

Among the most highly ranked GFINs by permutation testing, the MAX dimerization protein (MXD) GFIN (P_{Fisher}<=3.83×10⁻²³, Enrichment=2.53, Pₚₑᵣₘ<=0.042) was the most enriched. The MXD family of genes encode proteins that interact with MYC/MAX network of basic helix-loop-helix leucine zipper (bHLHZ) transcription factors that regulate cell proliferation, differentiation, and apoptosis [MIM 600021] [57]; MXD genes are important candidate tumor suppressor genes as the MXD-MYC-MAX network is dysregulated in various types of cancer [58]. Interestingly an epidemiological link between autism and specific types of cancer has been reported [59] and anti-cancer therapeutics were recently shown to modulate ASD phenotypes in the mouse through regulation of synaptic NLGN protein levels [60]. Within the component genes contributing to the MXD GFIN significance, duplications in PARP10 (P<=4.06×10⁻¹¹, OR=2.04) and UBE3A (1.50×10⁻⁶, OR=inf) are the most significantly enriched (data not shown). It is notable that we found PARP8 as significant across ethnicities as described earlier (Table 6), and we previously described the importance of structural defects in UBE3A in the ASDs [23].

Other notable significant GFINs uncovered were POU class 5 homeobox (POU5F) GIFN (P_{Fisher}<=2.96×10⁻¹⁷, Enrichment=2.3, Pₚₑᵣₘ<=0.008, and the SWI/SNF related, matrix associated, actin dependent regulator of chromatin, subfamily c (SMARCC) GFIN (P_{Fisher}<=1.22×10⁻⁹, Enrichment=1.9, Pₚₑᵣₘ, <=0.035). The POU5F family of genes encodes for transcription factors containing a POU homeodomain, and their role has been demonstrated in embryonic development, especially during early embryogenesis, and it is necessary for embryonic stem cell pluripotency. Component genes of the SMARCC gene family are members of the SWI/SNF family of proteins, whose members display helicase and ATPase activities and which are thought to regulate transcription of certain genes by altering the chromatin structure around those genes. Most interestingly, the KIAA family of genes ranked among the top GFINs (P_{Fisher}<=3.12×10⁻²³, Enrichment=1.6, Pₚₑᵣₘ<=0.040). KIAA genes have been identified in the Kazusa cDNA sequencing project [61], and are predicted from novel large human cDNAs; however, they have no known function.

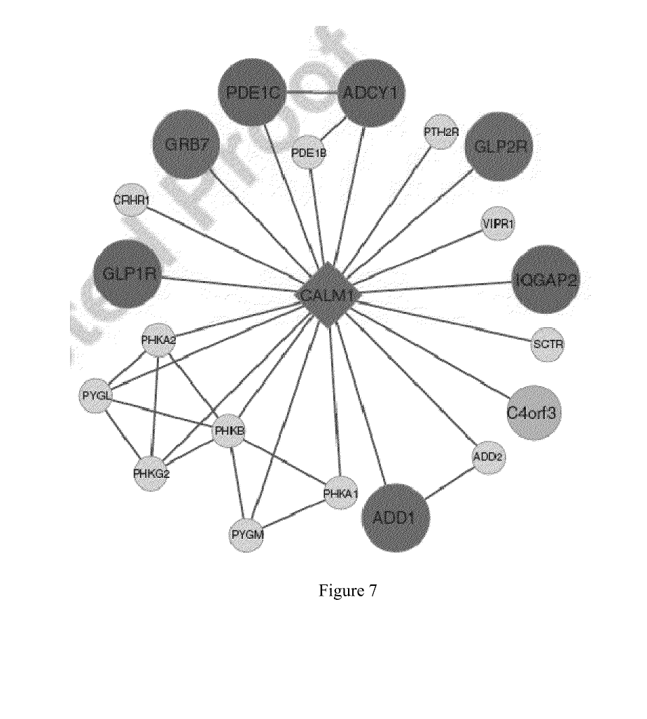

We also hypothesized that some component members of gene families may contribute disproportionately to the significance of a GFIN because they are highly connected to interacting gene partners that are enriched for CNV defects in ASD. Therefore we decomposed the 1,732 gene families into their 15,352 component duplicated genes of which 1,218 had defined networks with data to test for significance by genome-wide network permutation. The calmodulin 1 (CALM 1) gene interaction network ranked highest by network permutation testing of case enrichment for CNV defects across 1,000 random gene networks (Table 8, FIG. 7) and represents a novel and attractive candidate gene for the ASDs. Across the CALM1 network, we found CNV defects in 14/4618 cases vs only 1/4726 controls (P_{fisher}<=4.16×10⁴, Enrichment=14.37, Pₚₑᵣₘ<=0.002), and these defects were distributed such that 90% (9/10) of genes that harbored CNVs in the CALM1 interactome were enriched in cases. Closer inspection of the most significant CNVR contributing to the CALM1 network significance (Data not shown) revealed that no single gene was significant on its own; instead, with the exception of only one gene (PTH2R), each contributing CNVR tagged highly penetrant rare defects unique to cases. Calmodulin is the archetype of the family of calcium-modulated proteins of which nearly 20 members have been found. Calmodulin contains 149 amino acids that define 4 calcium-binding domains used for Ca²⁺-mediated coordination of a large number of enzymes, ion channels and other proteins including kinases and phosphatases; its functions include roles in growth and cell cycle regulation as well as in signal transduction and the synthesis and release of neurotransmitters [MIM 114180] [57].

Among other highly ranked first degree gene interaction networks, were the nuclear receptor co-repressor 1 (NCOR1; P_{fisher}<=1.11×10⁻⁶, Enrichment=13.37, Pₚₑᵣₘ<=0.004) and BCL2-associated athanogene 1 (BAG 1; P_{fisher}<=2.18×10⁴, Enrichment=15.40, Pₚₑᵣₘ<=0.014) networks. NCOR1 is a transcriptional co-regulatory protein that appears to assist nuclear receptors in the down regulation of DNA expression through recruitment of histone deacetylases to DNA promoter regions; it is a principal regulator in neural stem cells [51]. The oncogene BCL2 is a membrane protein that blocks the apoptosis pathway, and BAG1 forms a BCL2-associated athanogene and represents a link between growth factor receptors and anti-apoptotic mechanisms. The BAG1 gene has been implicated in age related neurodegenerative diseases, including Alzheimer's disease [62,63].

In summary, the private nature of mutations in the ASDs, and the cumulative contributions of rare highly penetrant genetic defects boost our power to discover and prioritize significant pathway defects. As a result, our comprehensive, unbiased analytical approach has identified a diverse set of specific defective biological pathways that contribute to the underlying etiology of the ASDs. Among GFINs robustly enriched for structural defects, the most enriched was that of the MXD family of genes that has been implicated in cancer pathogenesis [58] thereby providing concrete genetic defects to explore the reported coincidence of specific cancers with the ASDs [59]. The most highly ranked component duplicated gene interaction network involves defects in CALM1 and its multiple interacting partners that are important in regulating voltage independent calcium-activated action potentials at the neuronal synapse. Moreover, we found significant enrichment for defects within the GFIN for GRM that defines the mGluR pathway that has previously been shown to be defective in other neuropsychiatric diseases [29,30]. While specific mGluR gene family members have been shown to underlie syndromic ASDs [55], our findings suggest that rare defects in mGluR signaling also contribute to idiopathic autism across the entire GFIN for GRM genes.

Consequently, in addition to specific neuronal pathways that are expected to be defective in the ASDs like those defined by GRM and CALM duplicate genes, we implicate completely novel biological pathways such as the MXD pathway specific forms which appear to be associated with the ASDs [59]. Given the unmet need for better treatment for neurodevelopmental diseases [64], the functionally diverse set of defective genetic interaction networks we report presents attractive genetic biomarkers for targeted therapeutic intervention in ASDs and across the neuropsychiatric disease spectrum.

### REFERENCES FOR EXAMPLE III

1. Muhle R, Trentacoste S V, Rapin I (2004) The genetics of autism. Pediatrics 113: e472-86. Available: http://www.ncbi.nlm.nih.gov/pubmed/15121991. Accessed 4 Jun. 2011.
2. Fombonne E (2003) The prevalence of autism. JAMA 289: 87-89. Available: http://www.ncbi.nlm.nih.gov/entrez/query.fcgi?cmd=Retrieve&db=PubMed&dopt=Citati on&list_uids=12503982.
3. Prevalence of autism spectrum disorders—autism and developmental disabilities monitoring network, 14 sites, United States, 2002. (2007). MMWR Surveill Summ Morb Mortal Wkly report Surveill Summ/CDC 56: 12-28. Available: http://www.ncbi.nlm.nih.gov/pubmed/17287715. Accessed 6 Jun. 2011.
4. Prevalence of autism spectrum disorders—Autism and Developmental Disabilities Monitoring Network, United States, 2006. (2009). MMWR Surveill Summ Morb Mortal Wkly report Surveill Summ/CDC 58: 1-20. Available: http://www.ncbi.nlm.nih.gov/pubmed/20023608. Accessed 21 Feb. 2011.
5. Blumberg S J, Ph D, Bramlett M D (2013) Changes in Prevalence of Parent-reported Autism Spectrum Disorder in School-aged U.S. Children: 2007 to 2011-2012. Hyattsville, Md. 20782. Available: http://www.cdc.govinchs/data/nhsenhsr065.pdf.
6. Kim Y S, Leventhal B L, Koh Y-J, Fombonne E, Laska E, et al. (2011) Prevalence of Autism Spectrum Disorders in a Total Population Sample. Am J Psychiatry. Available: http://www.ncbi.nlm.nih.gov/pubmed/21558103. Accessed 11 May 2011.
7. Folstein S E, Rosen-Sheidley B (2001) Genetics of autism: complex aetiology for a heterogeneous disorder. Nat Rev Genet 2: 943-955. Available: http://www.ncbi.nlm.nih.gov/pubmed/11733747. Accessed 6 Jun. 2011.
8. Folstein S, Rutter M (1977) Infantile autism: a genetic study of 21 twin pairs. J Child Psychol Psychiatry 18: 297-321. Available: http://www.ncbi.nlm.nih.gov/pubmed/562353. Accessed 16 May 2011.
9. Steffenburg S, Gillberg C, Hellgren L, Andersson L, Gillberg I C, et al. (1989) A twin study of autism in Denmark, Finland, Iceland, Norway and Sweden. J Child Psychol Psychiatry 30: 405-416. Available: http://www.ncbi.nlm.nih.gov/pubmed/2745591. Accessed 7 Feb. 2011.
10. Bailey A, Le Couteur A, Gottesman I, Bolton P, Simonoff E, et al. (1995) Autism as a strongly genetic disorder: evidence from a British twin study. Psychol Med 25: 63-77. Available: http://www.ncbi.nlm.nih.gov/pubmed/7792363. Accessed 11 Jan. 2011
11. Ozonoff S, Young G S, Carter A, Messinger D, Yirmiya N, et al. (2011) Recurrence risk for autism spectrum disorders: a baby siblings research consortium study. Pediatrics 128: e488-95. Available: http://www.pubmedcentral.nih.gov/articlerender.fcgi?artid=3164092&tool=pmcentrez&rendertype=abstract. Accessed 3 Mar. 2012.
12. Constantino J N, Zhang Y, Frazier T, Abbacchi A M, Law P (2010) Sibling recurrence and the genetic epidemiology of autism. Am J Psychiatry 167: 1349-1356. Available: http://www.pubmedcentral.nih.gov/articlerender.fcgi?artid=2970737&tool=pmcentrez&rendertype=abstract. Accessed 12 Mar. 2012.
13. Kolevzon A, Smith C J, Schmeidler J, Buxbaum J D, Silverman J M (2004) Familial symptom domains in monozygotic siblings with autism. Am J Med Genet B Neuropsychiatr Genet 129B: 76-81. Available: http://www.ncbi.nlm.nih.gov/pubmed/15274045. Accessed 6 Jun. 2011.
14. Betancur C (2011) Etiological heterogeneity in autism spectrum disorders: more than 100 genetic and genomic disorders and still counting. Brain Res 1380: 42-77. Available: http://www.ncbi.nlm.nih.gov/pubmed/21129364. Accessed 14 Jul. 2012.
15. Iossifov I, Ronemus M, Levy D, Wang Z, Hakker I, et al. (2012) De novo gene disruptions in children on the autistic spectrum. Neuron 74: 285-299. Available: http://www.ncbi.nlm.nih.gov/pubmed/22542183. Accessed 16 Jul. 2012.
16. Huguet G, By E, Bourgeron T (2013) The genetic landscapes of autism spectrum disorders. Annu Rev Genomics Hum Genet 14: 191-213. Available: http://www.annualreviews.org/doi/abs/10.1146/annurev-genom-091212-153431. Accessed 23 Jan. 2014.
17. Sanders S J, Murtha M T, Gupta A R, Murdoch J D, Raubeson M J, et al. (2012) De novo mutations revealed by whole-exome sequencing are strongly associated with autism. Nature 485: 237-241. Available: http://www.ncbi.nlm.nih.gov/pubmed/22495306. Accessed 31 Jan. 2013.
18. Neale B M, Kou Y, Liu L, Ma'ayan A, Samocha K E, et al. (2012) Patterns and rates of exonic de novo mutations in autism spectrum disorders. Nature 485: 242-245. Available: http://www.ncbi.nlm.nih.gov/pubmed/22495311. Accessed 31 Jan. 2013.
19. O'Roak B J, Vives L, Girirajan S, Karakoc E, Krumm N, et al. (2012) Sporadic autism exomes reveal a highly interconnected protein network of de novo mutations. Nature 485: 246-250. Available: http://www.pubmedcentral.nih.gov/articlerender.fcgi?artid=3350576&tool=pmcentrez&rendertype=abstract. Accessed 30 Jan. 2013.
20. Jamain S, Quach H, Betancur C, Råstam M, Colineaux C, et al. (2003) Mutations of the X-linked genes encoding neuroligins NLGN3 and NLGN4 are associated with autism. Nat Genet 34: 27-29. Available: http://www.pubmedcentral.nih.gov/articlerender.fcgi?artid=1925054&tool=pmcentrez&rendertype=abstract. Accessed 26 Apr. 2011.
21. Pinto D, Pagnamenta A T, Klei L, Anney R, Merico D, et al. (2010) Functional impact of global rare copy number variation in autism spectrum disorders. Nature 466: 368-372. Available: http://www.ncbi.nlm.nih gov/pubmed/20531469. Accessed 15 Jul. 2010.
22. Szatmari P, Paterson A D, Zwaigenbaum L, Roberts W, Brian J, et al. (2007) Mapping autism risk loci using genetic linkage and chromosomal rearrangements. Nat Genet 39: 319-328. Available: http://www.ncbi.nlm.nih.gov/pubmed/17322880. Accessed 21 May 2011.
23. Glessner J T, Wang K, Cai G, Korvatska 0, Kim C E, et al. (2009) Autism genome-wide copy number variation reveals ubiquitin and neuronal genes. Nature 459: 569-573. Available: http://www.ncbi.nlm.nih.gov/pubmed/19404257. Accessed 14 Jul. 2010.
24. Buxbaum J D, Silverman J M, Smith C J, Greenberg D A, Kilifarski M, et al. (2002) Association between a GABRB3 polymorphism and autism. Mol Psychiatry 7: 311-316. Available: http://www.ncbi.nlm.nih.gov/pubmed/11920158. Accessed 7 Oct. 2011.
25. Collins A L, Ma D, Whitehead P L, Martin E R, Wright H H, et al. (2006) Investigation of autism and GABA receptor subunit genes in multiple ethnic groups. Neurogenetics 7: 167-174. Available: http://www.pubmedcentral.nih.gov/articlerender.fcgi?artid=1513515&tool=pmcentrez&rendertype=abstract. Accessed 28 Oct. 2010.
26. Ma D Q, Whitehead P L, Menold M M, Martin E R, Ashley-Koch a E, et al. (2005) Identification of significant association and gene-gene interaction of GABA receptor subunit genes in autism. Am J Hum Genet 77: 377-388. Available: http://www.pubmedcentral.nih.gov/articlerender.fcgi?artid=1226204&tool=pmcentrez&rendertype=abstract.
27. Matsunami N, Hadley D, Hensel C H, Christensen G B, Kim C, et al. (2013) Identification of Rare Recurrent Copy Number Variants in High-Risk Autism Families and Their Prevalence in a Large ASD Population. PLoS One 8: e52239. Available: http://www.pubmedcentral.nih.gov/articlerender.fcgi?artid=3544904&tool=pmcentrez&rendertype=abstract. Accessed 2 Feb. 2013.
28. Wang K, Zhang H, Ma D, Bucan M, Glessner J T, et al. (2009) Common genetic variants on 5p14.1 associate with autism spectrum disorders. Nature 459: 528-533. Available: http://www.pubmedcentral.nih.gov/articlerender.fcgi?artid=2943511&tool=pmcentrez&rendertype=abstract. Accessed 2 Apr. 2011.
29. Glessner J T, Reilly M P, Kim C E, Takahashi N, Albano A, et al. (2010) Strong synaptic transmission impact by copy number variations in schizophrenia. Proc Natl Acad Sci USA 107: 10584-10589. Available: http://www.pubmedcentral.nih.gov/articlerender.fcgi?artid=2890845&tool=pmcentrez&rendertype=abstract. Accessed 28 Feb. 2012.
30. Elia J, Glessner J T, Wang K, Takahashi N, Shtir C J, et al. (2012) Genome-wide copy number variation study associates metabotropic glutamate receptor gene networks with attention deficit hyperactivity disorder. Nat Genet 44: 78-84. Available: http://www.ncbi.nlm.nih.gov/pubmed/22138692. Accessed 29 Jan. 2013.
31. Fujii Y, Shibata H, Kikuta R, Makino C, Tani A, et al. (2003) Positive associations of polymorphisms in the metabotropic glutamate receptor type 3 gene (GRM3) with schizophrenia. Psychiatr Genet 13: 71-76. Available: http://www.ncbi.nlm.nih.gov/pubmed/12782962. Accessed 24 Mar. 2014.
32. Li Z-J, Wang B-J, Ding M, Pang H, Sun X-F, et al. (2008) [The association between glutamate receptor gene SNP and schizophrenia]. Fa Yi Xue Za Zhi 24: 369-374,377. Available: http://www.nebi.nlm.nih.gov/pubmed/18979923. Accessed 24 March 2014.
33. Shibata H, Tani A, Chikuhara T, Kikuta R, Sakai M, et al. (2009) Association study of polymorphisms in the group III metabotropic glutamate receptor genes, GRM4 and GRM7, with schizophrenia. Psychiatry Res 167: 88-96. Available: http://www.ncbi.nlm.nih.gov/pubmed/19351574. Accessed 24 Mar. 2014.
34. Ohtsuki T, Koga M, Ishiguro H, Horiuchi Y, Arai M, et al. (2008) A polymorphism of the metabotropic glutamate receptor mGluR7 (GRM7) gene is associated with schizophrenia. Schizophr Res 101: 9-16. Available: http://www.ncbi.nlm.nih.gov/pubmed/18329248. Accessed 24 Mar. 2014.
35. Bolonna A A, Kerwin R W, Munro J, Arranz M J, Makoff A J (2001)
Polymorphisms in the genes for mGluR types 7 and 8: association studies with schizophrenia. Schizophr Res 47: 99-103. Available: http://www.sciencedirect.com/science/article/pii/S0920996499002352. Accessed 24 Mar. 2014.
36. Takaki H, Kikuta R, Shibata H, Ninomiya H, Tashiro N, et al. (2004) Positive associations of polymorphisms in the metabotropic glutamate receptor type 8 gene (GRM8) with schizophrenia. Am J Med Genet B Neuropsychiatr Genet 128B: 6-14. Available: http://www.ncbi.nlm.nih.gov/pubmed/15211621. Accessed 24 Mar. 2014.
37. Moreno-De-Luca D, Sanders S J, Willsey A J, Mulle J G, Lowe J K, et al. (2013) Using large clinical data sets to infer pathogenicity for rare copy number variants in autism cohorts. Mol Psychiatry 18: 1090-1095. Available: http://dx.doi.org/10.1038/mp.2012.138. Accessed 10 Feb. 2014.
38. Gilman S R, Iossifov I, Levy D, Ronemus M, Wigler M, et al. (2011) Rare de novo variants associated with autism implicate a large functional network of genes involved in formation and function of synapses. Neuron 70: 898-907. Available: http://www.cell.com/neuron/fulltext/S0896-6273(11)00439-9. Accessed 23 Jan. 2014.
39. Sakai Y, Shaw C A, Dawson B C, Dugas D V, Al-Mohtaseb Z, et al. (2011) Protein interactome reveals converging molecular pathways among autism disorders. Sci Transl Med 3: 86ra49. Available: http://stm.sciencemag.org/content/3/86/86ra49. Accessed 21 Feb. 2014.
40. Noh H J, Ponting C P, Boulding H C, Meader S, Betancur C, et al. (2013) Network topologies and convergent aetiologies arising from deletions and duplications observed in individuals with autism. PLoS Genet 9: e1003523. Available: /pmcc/articles/PMC3675007/?report=abstract. Accessed 28 Feb. 2014.
41. Hopkins A L, Groom C R (2002) The druggable genome. Nat Rev Drug Discov 1: 727-730. Available: http://www.ncbi.nlm.nih.gov/pubmed/12209152. Accessed 22 January 2014.
42. Hadley D, Murphy T, Valladares 0, Hannenhalli S, Ungar L, et al. (2006) Patterns of sequence conservation in presynaptic neural genes. Genome Biol 7: R105. Available: http://www.pubmedcentral.nih.gov/articlerender.fcgi?artid=1794582&tool=pmcentrez&rendertype=abstract. Accessed 7 Feb. 2013.
43. Zoghbi H Y (2003) Postnatal neurodevelopmental disorders: meeting at the synapse? Science 302: 826-830. Available: http://www.ncbi.nlm.nih.gov/pubmed/14593168. Accessed 29 Nov. 2010.
44. Anney R, Klei L, Pinto D, Regan R, Conroy J, et al. (2010) A genomewide scan for common alleles affecting risk for autism. Hum Mol Genet. Available: http://www.ncbi.nlm.nih.gov/pubmed/20663923. Accessed 30 Jul. 2010.
45. Altshuler D M, Gibbs R A, Peltonen L, Dermitzakis E, Schaffner S F, et al. (2010) Integrating common and rare genetic variation in diverse human populations. Nature 467: 52-58. Available: http://dx.doi.org/10.1038/nature09298. Accessed 18 Jul. 2011.
46. Cann H M, de Toma C, Cazes L, Legrand M, Morel V, et al. (2002) A human genome diversity cell line panel. Science 296: 261-262. Available: http://www.ncbi.nlm.nih.gov/pubmed/11954565. Accessed 14 Oct. 2010.
47. Van der Zwaag B, Franke L, Poot M, Hochstenbach R, Spierenburg H A, et al. (2009) Gene-network analysis identifies susceptibility genes related to glycobiology in autism. PLoS One 4: e5324. Available: http://www.plosone.org/article/info:doi/10.1371/journal.pone.0005324?imageURI=info:doi/10.1371/journal.pone.0005324.t001. Accessed 16 Nov. 2012.
48. HUGO Gene Nomenclature Committee Home Page IHUGO Gene Nomenclature Committee (n.d.). Available: http://www.genenames.org/. Accessed 18 Feb. 2013.
49. Venkatesan K, Rual J-F, Vazquez A, Stelzl U, Lemmens I, et al. (2009) An empirical framework for binary interactome mapping. Nat Methods 6: 83-90. Available: http://dx.doi.org/10.1038/nmeth.1280. Accessed 31 Jan. 2013.
50. Rual J-F, Venkatesan K, Hao T, Hirozane-Kishikawa T, Dricot A, et al. (2005) Towards a proteome-scale map of the human protein-protein interaction network. Nature 437: 1173-1178. Available: http://www.ncbi.nlm.nih.gov/pubmed/16189514. Accessed 6 July 2011.
51. Hermanson 0, Jepsen K, Rosenfeld M G (2002)N-CoR controls differentiation of neural stem cells into astrocytes. Nature 419: 934-939. Available: http://www.ncbi.nlm.nih.gov/pubmed/12410313. Accessed 18 Feb. 2013.
52. Li H, Li Y, Shao J, Li R, Qin Y, et al. (2008) The association analysis of RELN and GRM8 genes with autistic spectrum disorder in Chinese Han population. Am J Med Genet B Neuropsychiatr Genet 147B: 194-200. Available: http://www.ncbi.nlm.nih.gov/pubmed/17955477. Accessed 12 Mar. 2014.
53. Serajee F J, Zhong H, Nabi R, Huq AHMM (2003) The metabotropic glutamate receptor 8 gene at 7q31: partial duplication and possible association with autism. J Med
Genet 40: e42. Available: http://www.pubmedcentral.nih.gov/articlerendericgi?artid=1735437&tool=pmcentrez&rendertype=abstract. Accessed 12 Mar. 2014.
54. Cusco I, Medrano A, Gener B, Vilardell M, Gallastegui F, et al. (2009) Autism-specific copy number variants further implicate the phosphatidylinositol signaling pathway and the glutamatergic synapse in the etiology of the disorder. Hum Mol Genet 18: 1795-1804. Available: http://hmg.oxfordjournals.org/content/18/10/1795.long. Accessed 13 Feb. 2014.
55. Auerbach B D, Osterweil E K, Bear M F (2011) Mutations causing syndromic autism define an axis of synaptic pathophysiology. Nature 1. Available: http://www.nature.com/doifinder/10.1038/nature10658. Accessed 1 Dec. 2011.
56. Elia J, Gai X, Xie H M, Perin J C, Geiger E, et al. (2009) Rare structural variants found in attention-deficit hyperactivity disorder are preferentially associated with neurodevelopmental genes. Mol Psychiatry: 637-646. Available: http://www.ncbi.nlm.nih.gov/pubmed/19546859.
57. OMIM—Online Mendelian Inheritance in Man (n.d.). Available: http://omim.org/. Accessed 18 Feb. 2013.
58. Nair S K, Burley S K (2003) X-ray structures of Myc-Max and Mad-Max recognizing DNA. Molecular bases of regulation by proto-oncogenic transcription factors. Cell 112: 193-205. Available: http://www.ncbi.nlm.nih.gov/pubmed/12553908. Accessed 21 Feb. 2013.
59. Kao H-T, Buka S L, Kelsey K T, Gruber D F, Porton B (2010) The correlation between rates of cancer and autism: an exploratory ecological investigation. PLoS One 5: e9372. Available: http://www.pubmedcentral.nih.gov/articlerender.fcgi?artid=2826417&tool=pmcentrez&rendertype=abstract. Accessed 21 Feb. 2013.
60. Gkogkas C G, Khoutorsky A, Ran I, Rampakakis E, Nevarko T, et al. (2013) Autism-related deficits via dysregulated eIF4E-dependent translational control. Nature 493: 371-377. Available: http://dx.doi.org/10.1038/nature11628. Accessed 28 Jan. 2013.
61. Kikuno R, Nagase T, Nakayama M, Koga H, Okazaki N, et al. (2004) HUGE: a database for human KIAA proteins, a 2004 update integrating HUGEppi and ROUGE. Nucleic Acids Res 32: D502-4. Available: http://www.pubmedcentral.nih.gov/articlerender.fcgi?artid=308769&tool=pmcentrez&re ndertype=abstract. Accessed 8 Feb. 2013.
62. Elliott E, Tsvetkov P, Ginzburg I (2007) BAG-1 associates with Hsc70.Tau complex and regulates the proteasomal degradation of Tau protein. J Biol Chem 282: 37276-37284. Available: http://www.jbc.org/content/282/51/37276.short. Accessed 18 Feb. 2013.
63. Elliott E, Laufer 0, Ginzburg I (2009) BAG-1M is up-regulated in hippocampus of Alzheimer's disease patients and associates with tau and APP proteins. J Neurochem 109: 1168-1178. Available: http://www.ncbi.nlm.nih.gov/pubmed/19317853. Accessed 18 Feb. 2013.
64. McMahon F J, Insel T R (2012) Pharmacogenomics and personalized medicine in neuropsychiatry. Neuron 74: 773-776. Available: http://www.ncbi.nlm.nih.gov/pubmed/22681682. Accessed 8 Mar. 2013.
65. R Core Team (n.d.) R: A language and environment for statistical computing. Available: http://www.r-project.org/. Accessed 18 Feb. 2013.
66. Wang K, Li M, Hadley D, Liu R, Glessner J, et al. (2007) PennCNV: an integrated hidden Markov model designed for high-resolution copy number variation detection in whole-genome SNP genotyping data. Genome Res 17: 1665-1674. Available: http://www.pubmedcentral.nih.gov/articlerender.fcgi?artid=2045149&tool=pmcentrez&rendertype=abstract. Accessed 30 Jul. 2010.
67. Purcell S, Neale B, Todd-Brown K, Thomas L, Ferreira M A R, et al. (2007) PLINK: a tool set for whole-genome association and population-based linkage analyses. Am J Hum Genet 81: 559-575. Available: http://www.pubmedcentral.nih.gov/articlerender.fcgi?artid=1950838&tool=pmcentrez&zrendertype=abstract. Accessed 11 Feb. 2013.
68. Human Interactome Database (n.d.). Available: http://interactome.dfci.harvard.edu/H_sapiens/. Accessed 18 Feb. 2013.

### Example IV

### The Role of mGluR Copy Number Variation in Genetic and Environmental Forms of Syndromic Autism Spectrum Disorder

Abnormal signaling mediated through mGluR5 is involved in the pathophysiology of Autism Spectrum Disorder (ASD) in Fragile X Syndrome and Tuberous Sclerosis. However, the role of other mGluR associated network/signaling genes in syndromic ASD is unknown. To determine whether copy number variants (CNV'S) are enriched in syndromic ASD, microarrays were used to identify mGluR network CNV's in children with ASD. We set out to determine 1) whether rate of syndromic features vary between children with ASD with and without CNV's in mGluR network genes; and 2) whether “second hits” in mGluR network genes occur more often in children with ASD in children with 22q11.2 Deletion Syndrome (who all have haploinsufficiency of RANBP1, an mGluR network gene in the 22q11.2 region.

Individuals in our biorepository with parental report of ASD (n=6,452) were screened for parental consent to access clinical evaluations in the Electronic Health Record at the Children's Hospital of Philadelphia (n=539). Our syndromic comparison cohort included children with 22₈11.2 Deletion Syndrome with full access to past medical and neuropsychological evaluations (n=75), including those with diagnosis of ASD (n=25) and those with no concern for ASD (n=50).

Patient categorization (syndromic vs nonsyndromic) was done via blinded medical chart review in all mGluR positive and 100 randomly selected mGluR negative cases.

Our results, explained further hereinbelow show that 11.5% of ASD had mGluR CNV's vs. 3.2% in healthy controls (p<0.001). Syndromic ASD was more prevalent in children with mGluR CNVs (72% vs 16%, p<0.001). A comparison cohort of children with 22q11.2 Deletion Syndrome (n=25 with ASD, n=50 without ASD), all haploinsufficient for mGluR network gene RANBP1, was evaluated to determine whether “second hits” in mGluR network genes confer additional risk for ASD. 20% with 22q11.2DS+ASD had “second hits” in mGluR signaling genes vs 2% in 22q11.2DS-ASD (p<0.014). Conclusions: We propose that altered RANBP1 expression may provide a mechanistic link between ASD in 22q11.2DS, Thalidomide Embryopathy and Fetal Valproate Syndrome, providing a link for seemingly unrelated genetic and environmental forms of ASD.

The results suggest that CNV's in mGluR network genes, previously implicated in altered neurological development in Fragile X Syndrome and Tuberous Sclerosis, may link many other genetic and environmental forms of Autism Spectrum Disorder.

As discussed in the previous examples, Autism Spectrum Disorder (ASD) occurs in approximately 1/88 individuals and is characterized by impairment in social communication and repetitive interests and activities¹. Approximately 20% of cases occur in the context of an identifiable syndrome². Genetic syndromes with ASD are heterogeneous, including cytogenetically visible chromosomal alterations (e.g. Trisomy 21), microdeletion and microduplication syndromes (e.g. 22q11.2 deletion syndrome [22q11.2DS]; 22q11.2 duplication syndrome [22q11.2DupS]), and monogenic disorders (e.g. Fragile X Syndrome [FXS], Tuberous Sclerosis [TS])³⁻¹³. In addition, prenatal exposure to thalidomide, valproic acid, misoprostol, ethanol and maternal rubella infection, have been associated with an elevated risk of ASD¹⁴⁻¹⁹.

The mechanism for the development of ASD in most forms of idiopathic and syndromic forms of ASD remains elusive. Recently, signaling through metabotropic glutamate receptor 5 (mGluR5) has been implicated in the development of ASD in FXS and TS²⁰. In FXS, abnormal production of Fragile X Mental Retardation Protein (FMRP) removes normal inhibition of signaling through the mGluR pathway. Tuberous Sclerosis leads to over inhibition of signaling. Auerbach and colleagues (2011) demonstrated abnormal synaptic learning and atypical behavior in mouse models of FXS and TS, and reversed these effects by breeding the two strains together—mice harboring both mutations had normal mGluR signaling, and learning and behavior that was indistinguishable from control mice²⁰. Other studies have demonstrated normalization of learning and behavior in Fragile X mice by administration of an mGluR5 antagonist^{21,22}. In addition to elucidating the mechanism for cognitive and behavioral differences in FXS and TS, these studies suggest a promising avenue for pharmacological treatment. Recent studies have implicated rare CNV's in the etiology of ASD, including deletions impacting genes in the mGluR gene network²³, consisting of 276 genes²⁴. To determine whether additional forms of syndromic ASD may share a similar mechanism (through disruption of the mGluR gene network), we analyzed DNA from 539 children with ASD (not filtered for comorbid genetic syndrome) followed at the Children's Hospital of Philadelphia.

The following materials and methods are provided to facilitate the practice of Example IV.

### Participants:

Phenotypic data for patients with ASD as reported on parental health questionnaires from our biorepository (n=6,452) were evaluated to identify patients who received clinical assessment at the Children's Hospital of Philadelphia and agreed to Electronic Health Record chart review. DNA from these cases (n=539) were selected for further phenotypic and genotypic analysis. Children were recruited for inclusion in the general Center for Applied Genomics biorepository when they were getting blood drawn for another purpose at The Children's Hospital of Philadelphia, so there is an overrepresentation of children with at least one medical problem in this patient cohort. The parents of all patients gave consent for participation in the study, which was approved by the Institutional Review Board at the Children's Hospital of Philadelphia (IRB 06-004886).

### Chart Review:

Subject selection and randomization process: All patients with an mGluR CNV (n=62) and 100 patients without mGluR CNV were randomly selected for chart review. This procedure was selected to ensure that all patients with mGluR CNV received detailed chart review with an adequately sized comparison cohort. A three step process was done to ensure blinded chart review. The selection of the 162 charts was done by a geneticist with access to CNV data but without access to the Electronic Health Record (CK). Another author who had no access to CNV data nor the Electronic Health Record blinded and randomized the patient ID's (RTS). Finally, a physician with access to the Electronic Health Record but blinded to mGluR status (TLW) reviewed charts for documentation of ASD diagnosis and presence of other medical comorbidities.

### ASD:

Charts were reviewed to confirm a diagnosis of ASD and also to determine medical comorbidities for each patient. Diagnosis of ASD was confirmed in the chart, but as this was a retrospective chart review, gold-standard research instruments (e.g. Autism).

### Medical Comorbidities:

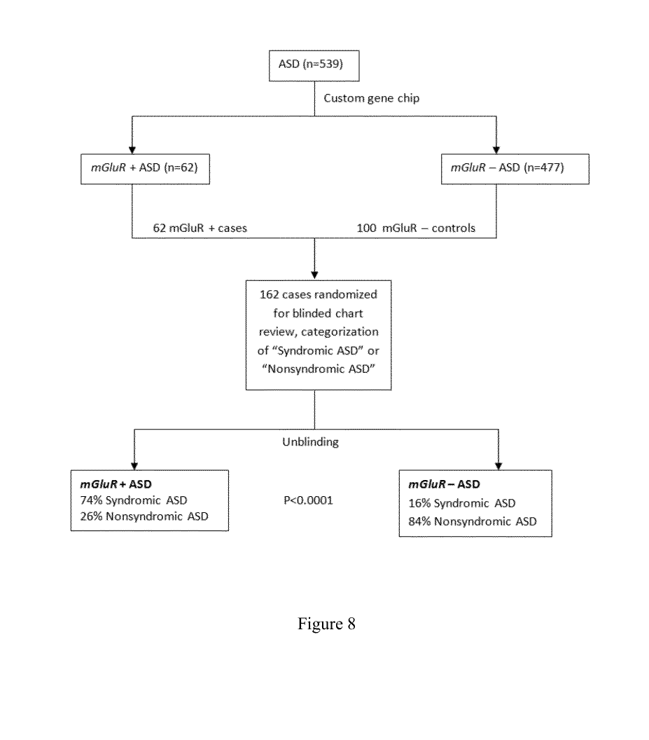

Structural birth defects, genetic testing and medical conditions were recorded for each patient. Cases were categorized as “Syndromic ASD” if they had ASD and presence of a medical condition orstructural birth defect (e.g. cleft palate) that occurs in less than 1% of the general population. This criteria was established to define a subset of patients whose ASD and other medical problems would be highly unlikely to occur coincidentally—With a baseline rate of ASD at 1/88 and a medical condition that occurs in <1% of the general population, the compound likelihood of both occurring by chance would be approximately 0.001%. See FIG. 8.

### Genotyping Arrays and CNV Calling:

DNA from subjects with ASD were each genotyped on the Human610-Quad or HumanHap550 SNP arrays from Illumina. For 22q11 DS cohorts, subjects were typed either on Illumina SNP arrays (Human610-Quad v1.0 or HumanHap550) or Affymetrix 6.0 SNP arrays. Clustering and SNP calling was performed using GenomeStudio (Illumina) to generate normalized intensity (i.e. Log-R ratio, or LRR) and B-allele frequencies (BAF). CNV calling was performed using the PennCNV algorithm [PMID: 17921354] following waviness correction [PMID: 18784189]. In brief, PennCNV uses a hidden Markov model (HMM) that incorporates information from LRR, BAF, as well as features of the array (e.g. distance between neighboring SNPs) to detect CNVs.

### CNV Quality Control:

Samples with SNP arrays of poor quality were excluded from CNV calling, since typically the proportion of false positives increases considerably for these samples. Those samples where the genotyping call rate>96%, standard deviation of LRR (LRR sd)<0.4, GC-wave factor (GCWF) is between −0.2 and 0.2 after waviness correction, and total number of CNV calls for the sample <100 were included in analysis.

### CNV Annotation:

For syndromic ASD regions, genomic coordinates were those described by Betancur [PMID: 21129364]. The GRM/mGluR network generated by Cytoscape from the Human Interactome database was described by Elia et al. [PMID: 22138692] using UCSC Genome Browser definitions for gene coordinates (UCSC genes). This network from Cytoscape was used to define mGluR+ vs. mGluR-subsets. For 22q11 DS cohort analysis, additional GRM/mGluR network genes were identified based on 1^{st} degree interaction network of the eight GRM genes using the program Ingenuity Pathway Analysis (Ingenuity Systems Inc./Qiagen; Redwood City, Calif.) as well as the genes encoding the group I mGluR signaling pathway described in Kelleher et al. [PMID: 22558107]. CNV calls were analyzed for overlap to known syndromic regions and GRM network genes. All syndromic aberrations detected by clinical cytogenetic laboratory testing were confirmed on corresponding SNP arrays.

### Results

### mGluR Network Copy Number Variations (CNVs) are Prevalent in Syndromic ASD Compared to Nonsyndromic ASD

CNVs in the mGluR network were found in 74% of patients with syndromic ASD compared to 16% of patients with nonsyndromic ASD (p<0.001). Most of the mGluR CNV's in patients with syndromic ASD (75%) were included in larger clinically significant CNV's. As mGluR network genes are present in the 22₈11.2 region (RANBP1) and on chromosome 21 (APP GRIK1 MX1 PCBP3 SETD4), patients with ASD in the presence of 22q11.2DS, 22q11.2DupS or Trisomy 21 accounted for 15 (33%) of the patients with Syndromic ASD+mGluR network changes. The remainder of observed cytogenetic changes had individual non-overlapping deletions or duplications. The analysis was repeated after exclusion of children with Trisomy 21, 22q11.2DS and 22q11.2DupS, (the syndromes in children in this study which have previously been associated with ASD). After their exclusion, the effect remained significant (p<0.001).

Autism Spectrum Disorder in 22q11.2 Deletion Syndrome is Associated with “Second Hit” in mGluR Pathway

As a comparison cohort, data from children with 22q11.2 DS with ASD (n=25) and without ASD (n=50) who had completed high density microarray evaluation (either Affymetrix 6.0, Illumina 500K, and Illumina 610Q) and clinical developmental assessments (as enrolled through a parallel study, approved by the Children's Hospital of Philadelphia Institutional Review Board, IRB 07-005352) were examined for the presence of a second mGluR network hit outside of the 22q11.2 region. “Second hits”, deletions of an mGluR network gene outside of the 22q11.2 region, were found in 20% (5/25) of patients with ASD and only 2% (1/50) without ASD (p<0.014).

### DISCUSSION

Prior studies have demonstrated that abnormal signaling (either too much or too little) through mGluR5 could be the basis for abnormal neural development (and possibly ASD) in FXS and TS. Our data suggest that derangement of the mGluR network may be responsible for increased rates of ASD seen in cytogenetically distinct forms of syndromic ASD. mGluR network genes are found in the 22q11.2 region as well as on Chromosome 21, which may be involved in the increased prevalence of ASD in both Down Syndrome and 22q11.2 DS. However, all patients with Trisomy 21 or 22q11.2 DS harbor the change in the mGluR network suggesting a second hit outside of the region may be necessary for expression of the ASD phenotype.

### Autism Spectrum Disorder in 22q11.2 Deletion Syndrome, 22q11.2 Duplication Syndrome, Thalidomide Embryopathy and Fetal Valproate Syndrome

The 22q11.2 DS is the most common microdeletion syndrome in humans, occurring in 1 in 4,000 individuals. The typical deletion spans approximately 3 Mb and includes approximately 45 genes, causing a variety of medical and behavioral disorders (Table 9)²⁵⁻²⁸. ASD occurs in approximately 20%, and psychosis in 25%^{5,9}. The 22q11.2DupS results in the same types of birth defects and medical comorbidities seen in 22q11.2 DS, but at a lower rate (among over 60 patients in our clinical cohort). There are no cases of psychosis in 22q11DupS in the literature²⁹ or our cohort. Among our cases with documentation of developmental evaluation after the age of 4, the prevalence of ASD is 27%, which is slightly higher than the rate in children with 22q11.2DS.

Thalidomide exposure during pregnancy causes a variety of birth defects that have all been reported in 22q11.2DS, including some that are extremely rare (e.g. phocomelia, radial ray defects). (Table 9). Miller and Stromland reported an elevated risk of ASD following exposure to thalidomide during early embryogenesis¹⁶. This study included prospective evaluation by a psychiatrist was done for adults who had been exposed to thalidomide during pregnancy and evaluation by a physician to document birth defects and associated features. All cases of ASD following thalidomide exposure had ear anomalies, suggesting exposure between days 24-28 post-fertilization. Among individuals exposed at this time, there was a 27% rate of ASD. Replications of this study in additional cohorts of children have not been possible because the use of thalidomide in pregnant women was widely restricted in the 1960's; therefore, additional cases are not available. Though several mechanisms for the cause of many of the birth defects in thalidomide embryopathy have been proposed, animal studies of the teratogenic effects of thalidomide have been limited due to significant species differences. One of the reasons thalidomide was used widely in the 1960's was because of a lack of teratogenicity in animals at levels that are highly teratogenic in humans. This has resulted in significant limitation in the ability of researchers to determine the teratogenic mechanism of thalidomide, as studies have taken place in animals for which thalidomide is not particularly teratogenic, or using dosages which are much higher than that used in humans. Recent changes in legislation have allowed for a study to be completed in human embryonic stem cells—the first of its kind to use human cells and dosages which would have been analogous to that experienced by women taking thalidomide in the 1950's and 1960's³⁰. This study, conducted by Meganathan and colleages (2012) proposed that the teratogenic effects of thalidomide may be mediated through RANBP1³⁰. Valproic acid (VPA) is widely used as an anticonvulsant, mood stabilizer, and to prevent migraine headaches. Exposure to VPA during pregnancy causes an increased rate of several birth defects, all of which have been reported in 22q11.2DS, and most of which have been seen in Thalidomide Embryopathy (Table 9). Table 9 compares the birth defects seen in 22q11.2 DS, Thalidomide Embryopathy and Fetal Valproate Syndrome. The comparison of all birth defects seen in 22q11.2 DS to the exposures syndromes was not made because 22q11.2 DS includes deletion of dozens of additional genes which we do not propose to be affected in Thalidomide Embryopathy or Fetal Valproate Syndrome. In addition to structural defects, children exposed to VPA in utero have an elevated risk of developing ASD^{19,31,32}. Rodent models of autism have used prenatal exposure to VPA to reproduce some of the neuroanatomic features of autism and abnormal behavior³³⁻³⁵. Due to its action as a Histone Deacetylase Inhibitor, VPA affects expression of many genes. Based on homology, decreased expression of RanBP1 mRNA is predicted in VPA-treated rats. Moreover, a recent study showed reversal of atypical behaviors in VPA-exposed mice with treatment with an mGluR antagonist³⁶.

TABLE 9
Birth defects seen in Fetal Valproate Syndrome and
Thalidomide Embryopathy are all reported in children
with 22q11.2 Deletion Syndrome

| | Fetal | | 22q11.2 |
|---|---|---|---|
| | Valproate | Thalidomide | Deletion |
| Birth Defect | Syndrome | Embryopathy | Syndrome |
| Autism | + | + | + |
| Cleft lip/palate | + | + | + |
| Congenital heart | + | + | + |
| disease | | | |
| Craniosynostosis | + | − | + |
| External Ear | + | + | + |
| anomalies | | | |
| Gastrointestinal tract | + | + | + |
| abnormalities (e.g. | | | |
| atresias) | | | |
| Genitourinary tract | + | + | + |
| anomalies (e.g. renal | | | |
| anomalies) | | | |
| Hypospadias | + | + | + |
| Neural tube defects | + | + | + |
| Ocular anomalies | + | + | + |
| (structural and ocular | | | |
| motility defects) | | | |
| Phocomelia | + | + | + |
| Polydactyly | + | + | + |
| Radial ray defects | + | + | + |
| Vertebral anomalies | + | + | + |

### CONCLUSION

Derangement of genes in the mGluR network are found at a high rate in patients with different forms of Syndromic ASD, including 22q11.2DS, 22q11.2DupS, Trisomy 21 and a large number of other seemingly-unrelated chromosomal alterations. Moreover, among children with 22q11.2DS, the presence of a “second hit” in the mGluR network was identified in 20% of children with ASD, and only 2% of those without ASD (p<0.014). Significantly, four children, all with autism phenotype, had a small deletion in the vicinity of the RANBP1 gene. While the expression level of RANBP1 was not affected in one individual available for testing (data not shown), these atypical deletions could impact gene function with resulting dysregulation of the RANBP1 protein.

Taken together, these data implicate dysregulation of the mGluR network as a likely permissive factor that increases the propensity to develop an ASD. The striking increase in prevalence of ASD with a CNV affecting a second gene in the network suggests perturbations of mGluR signaling at multiple points is necessary. It is important to note that CNV's represent only a fraction of changes in mGluR network genes, as this study did not include assessment of sequence variations, and these findings may therefore represent the “tip of the iceberg”. While perturbation of the mGluR network appears to confer risk of ASD, additional genetic or environmental stressors are likely necessary for an individual child to develop ASD.

Striking similarities exist in the profiles of birth defects and elevated rates of Autism Spectrum Disorder seen in 22q11.2 Deletion Syndrome, Fetal Valproate Syndrome and Thalidomide Embryopathy. As thalidomide and VPA both cause decreased expression of RANBP1 mRNA, mimicking haploinsufficiency of the gene in 22q11.2 Deletion Syndrome, it is plausible that it could be involved in the common teratogenic profile across syndromes. Moreover, results from a Ranbp1 knockout mouse model from Paronett et al³⁷ are also supportive of our hypothesis of the importance of RANBP1 in the neurological consequences of 22q11.2 DS and prenatal exposures affecting expression of RANBP1. In these studies, Ranbp1 (−/−) homozygotes, proliferation of the basal progenitor pool in the cortex is disrupted, leading to a dramatic reduction in cortical thickness and substantially fewer neurons in the perinatal cortex. The changes resulting from loss of RANBP1 function parallel that seen in mice with the larger 22q11.2 Deletion, suggesting that haploinsufficiency of Ranbp1 may contribute to the disruption of cortical circuitry in 22q11DS. Future studies, addressing the neurodevelopmental phenotype of mice with haploinsufficiency of Ranbp1 are anticipated to help elucidate the mechanism by which alterations of the mGluR pathway leads to increased risk of ASD,

### REFERENCES FOR EXAMPLE IV

1. Prevalence of Autism Spectrum Disorders—Autism and Developmental Disabilities Monitoring Network, 14 Sites, United States, 2008. at <http://www.cdc.gov/mmwr/preview/mmwrhtml/ss6103a1.htm?s_cid=ss6103a1_w>
2. Gurrieri, F. Working up autism: the practical role of medical genetics. in Am. J. Med. Genet. C Semin. Med. Genet. 160, 104-110 (2012).
3. Christian, S. L. et al. Novel submicroscopic chromosomal abnormalities detected in autism spectrum disorder. Biol. Psychiatry 63, 1111 (2008).
4. Gillberg, C. Chromosomal disorders and autism. J. Autism Dev. Disord. 28, 415-425 (1998).
5. Fine, S. E. et al. Autism spectrum disorders and symptoms in children with molecularly confirmed 22q11.2 deletion syndrome. J. Autism Dev. Disord. 35, 461-470 (2005).
6. Lo-Castro, A. et al. Association of syndromic mental retardation and autism with 22q11.2 duplication. Neuropediatrics 40, 137-140 (2009).
7. Motavalli Mukaddes, N. & Herguner, S. Autistic disorder and 22q11.2 duplication. World J. Biol. Psychiatry 8, 127-130 (2007).
8. Ramelli, G. P. et al. Microduplication 22q11. 2 in a child with autism spectrum disorder: clinical and genetic study. Dev. Med. Child Neurol. 50, 953-955 (2008).
9. Vorstman, J. A. et al. The 22q11.2 deletion in children: high rate of autistic disorders and early onset of psychotic symptoms. J. Am. Acad. Child Adolesc. Psychiatry 45, 1104-1113 (2006).
10. Mazzocco, M. M., Kates, W. R., Baumgardner, T. L., Freund, L. S. & Reiss, A. L. Autistic behaviors among girls with fragile X syndrome. J. Autism Dev. Disord. 27, 415-435 (1997).
11. Brown, W. T. et al. Fragile X and autism: a multicenter survey. Am. J. Med.
Genet. 23, 341-352 (1986).
12. Gillberg, J. C., Gillberg, C. & Ahlsén, G. Autistic behaviour and attention deficits in tuberous sclerosis: a population-based study. Dev. Med. Child Neurol. 36, 50-56 (1994).
13. Jeste, S. S., Sahin, M., Bolton, P., Ploubidis, G. B. & Humphrey, A. Characterization of autism in young children with tuberous sclerosis complex. J. Child Neurol. 23, 520-525 (2008).
14. Nanson, J. L. Autism in fetal alcohol syndrome: a report of six cases. Alcohol. Clin. Exp. Res. 16, 558-565 (1992).
15. Bandim, J. M., Ventura, L. 0., Miller, M. T., Almeida, H. C. & Costa, A. E. S. Autism and Möbius sequence: an exploratory study of children in northeastern Brazil. Arq. Neuropsiquiatr. 61, 181-185 (2003).
16. Stromland, K., Nordin, V., Miller, M., Akerstrom, B. & Gillberg, C. Autism in thalidomide embryopathy: a population study. Dev. Med. Child Neurol. 36, 351-356 (1994).
17. Chess, S. Autism in children with congenital rubella. J Autism Child. Schizophr. 1, 33-47 (1971).
18. Chess, S. Follow-up report on autism in congenital rubella. J. Autism Child Schizophr. 7, 69-81 (1977).
19. Christensen, J. et al. Prenatal Valproate Exposure and Risk of Autism Spectrum Disorders and Childhood AutismPrenatal Valproate and Autism. JAMA 309, 1696-1703 (2013).
20. Auerbach, B. D., Osterweil, E. K. & Bear, M. F. Mutations causing syndromic autism define an axis of synaptic pathophysiology. Nature 480, 63-68 (2011).
21. Thomas, A. M., Bui, N., Perkins, J. R., Yuva-Paylor, L. A. & Paylor, R. Group I metabotropic glutamate receptor antagonists alter select behaviors in a mouse model for fragile X syndrome. Psychopharmacology (Berl.) 219, 47-58 (2012).
22. Choi, C. H. et al. Pharmacological reversal of synaptic plasticity deficits in the mouse model of fragile X syndrome by group II mGluR antagonist or lithium treatment. Brain Res. 1380, 106-119 (2011).
23. Gai, X. et al. Rare structural variation of synapse and neurotransmission genes in autism. Mol. Psychiatry 17, 402-411 (2011).
24. Elia, J. et al. Genome-wide copy number variation study associates metabotropic glutamate receptor gene networks with attention deficit hyperactivity disorder. Nat. Genet. 44, 78-84 (2011).
25. Bassett, A. S. et al. Practical Guidelines for Managing Patients with 22q11.2 Deletion Syndrome. J. Pediatr. 159, 332-9.e1 (2011).
26. Goodship, J., Cross, I., LiLing, J. & Wren, C. A population study of chromosome 22q11 deletions in infancy. Arch. Dis. Child 79, 348-351 (1998).
27. Du Montcel, S. T., Mendizabai, H., Ayme, S., Levy, A. & Philip, N. Prevalence of 22q11 microdeletion. J. Med. Genet. 33, 719 (1996).
28. Oskarsdottir, S., Vujic, M. & Fasth, A. Incidence and prevalence of the 22q11 deletion syndrome: a population-based study in Western Sweden. Arch. Dis. Child. 89, 148-151 (2004).
29. Brunet, A. et al. Failure to detect the 22q11.2 duplication syndrome rearrangement among patients with schizophrenia. Behav. Brain Fund. 4, (2008).
30. Meganathan, K. et al. Identification of thalidomide-specific transcriptomics and proteomics signatures during differentiation of human embryonic stem cells. Plos One 7, e44228 (2012).
31. Rasalam, A. D. et al. Characteristics of fetal anticonvulsant syndrome associated autistic disorder. Dev. Med. Child Neurol. 47, 551-555 (2005).
32. Moore, S. J. et al. A clinical study of 57 children with fetal anticonvulsant syndromes. J. Med. Genet. 37, 489-497 (2000).
33. Ingram, J. L., Peckham, S. M., Tisdale, B. & Rodier, P. M. Prenatal exposure of rats to valproic acid reproduces the cerebellar anomalies associated with autism. Neurotoxicol. Teratol. 22, 319-324 (2000).
34. Tomasz Schneider, R. P. Behavioral alterations in rats prenatally exposed to valproic acid: animal model of autism. Neuropsychopharmacology 30, 80-89 (2004).
35. Rodier, P. M., Ingram, J. L., Tisdale, B., Nelson, S. & Romano, J. Embryological origin for autism: developmental anomalies of the cranial nerve motor nuclei. J. Comp. Neurol. 370, 247-261 (1996).
36. Mehta, M. V., Gandal, M. J. & Siegel, S. J. mGluR5-antagonist mediated reversal of elevated stereotyped, repetitive behaviors in the VPA model of autism. Plos One 6, e26077 (2011).
37. Paronett, E. M., Meechan, D., Karpinsky, B. A., LaMantia, A-S., Maynard (T. M.) Submitted, personal communication. Ranbp1, deleted in DiGeorge/22q11.2 deletion syndrome, is a microcephaly gene.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1-12. (canceled)

13. A kit for use in a method for detecting a propensity for developing autism or autistic spectrum disorder, the kit comprising:
a) providing a plurality of nucleic acids comprising CNVs present in autistic or ASD patients identified using genome wide association studies of large patient cohorts, said CNVs being listed in Table II;
a) means for obtaining a sample from a patient and isolating nucleic acid therefrom;
b) reagents suitable for identifying the presence or absence of at least one deletion containing CNV in a target polynucleotide and means for creating a report from from said identifying.

14. A kit as claimed in claim 13, wherein the target polynucleotide is amplified prior to detection.

15. The kit of claim 13, wherein the step of identifying the presence of said CNV further comprises reagents suitable for analyzing said nucleic acid by performing a process selected from the group consisting of detection of specific hybridization, measurement of allele size, restriction fragment length polymorphism analysis, allele-specific hybridization analysis, single base primer extension reaction, and sequencing of an amplified polynucleotide.

16. The kit as claimed in claim 13, containing reagents suitable for isolating DNA from said sample.

17. The kit of claim 13, comprising reagents for isolating CNV containing polynucleotides from an isolated cell of the human subject.

18. A method for identifying agents which alter neuronal signaling and/or morphology, comprising:
a) providing cells expressing a nucleic acid sequence comprising at least one CNV as claimed in claim 13;
b) providing cells which express the cognate wild type sequences corresponding to the CNV of step a);
c) contacting the cells of steps a) and b) with a test agent and
d) analyzing whether said agent alters neuronal signaling and/or morphology of cells of step a) relative to those of step b), thereby identifying agents which alter neuronal signaling and morphology.

19. A method of treating autism or ASD in a human subject determined to have at least copy number variation (CNV) associated with an autistic or ASD phenotype, said at least one CNV being selected from the group consisting of CNVs set out in Table 2, the method comprising administering to said human subject a therapeutically effective amount of at least one agent which is known to be efficacious in the signaling pathway adversely affected by the presence of said CNV.

20. The method of claim 19, wherein said CNV containing gene is selected from the group consisting of ATP10A, GABRA5, GABRB3, GABRG3, GGTLC2, HBII-52-45, HBII-52-46, IPW, LOC648691, LOC96610, MAGEL2, MIR650, MKRN3, NCRNA00221, NDN, OCA2, OR4S2, PAR-SN, PAR1, PARS, POM121L1P, PRAME, SNORD107, SNORD108, SNORD109A, SNORD109B, SNORD115-11, SNORD115-29, SNORD115-36, SNORD115-43, SNORD115-44, SNORD115-48, SNORD64, SNRPN, SNURF, UBE3A, ZNF280A, ZNF280B.

21. The method of claim 19, wherein said agent alters GABA signaling and is listed in Table 3 or Table 4.

22. The method of claim 21, wherein said gene is selected from the group consisting of GABRA5, GABRA3, GABRB3 and said agent is topiramide.

23. A kit for detecting a propensity for developing autism or autistic spectrum disorder, the kit comprising:
a) reagents suitable for obtaining a sample from a patient and isolating nucleic acid therefrom;
b) reagents suitable for amplifying the nucleic acid of step a);
c) a microarray comprising nucleic acids of known sequence with the amplified nucleic acids of step b), thereby identifying the presence or absence of at least one deletion containing CNV in a target polynucleotide, said CNV being listed in Table II; and means for creating a report indicating that if said CNV is present, said patient has an increased risk for developing autism and/or autistic spectrum disorder.
